(19) United States
(12) Patent Application Publication
(10) Pub. No.: US 2003/0232356 A1
(43) Pub. Date: Dec. 18, 2003

(54) Skin cell biomarkers and methods for identifying biomarkers using nucleic acid microarrays

(76) Inventors: Thomas P. Dooley, Vestavia Hills, AL (US); Ernest V. Curto, Huntsville, AL (US); Richard L. Davis JR., Homewood, AL (US)

Correspondence Address: Morrison & Foerster, LLP, Suite 300, 1650 Tysons Blvd., McLean, VA, 22102, US

(21) Appl. No.: 10/361,006

(22) Filed: Feb. 10, 2003

Related U.S. Application Data
(60) Provisional application No. 60/354,519, filed on Feb. 8, 2002.

(51) Int. Cl. C12Q 1/68; G06F 19/00; G01N 33/48; G01N 33/50

(52) U.S. Cl. 435/6; 702/20

(57) ABSTRACT

The present invention provides biomarker genes of mammalian skin-derived cells. A plurality of differentially-expressed up-regulated (signature) and down-regulated (anti-signature) biomarker genes for human keratinocytes, melanocytes, and fibroblasts are identified. Biomarker genes for cells at abnormal states such as melanoma cells are also provided. Further, there are provided analytical bioinformatic methods for identifying biomarker genes based on nucleic acid microarray data.

### REFERENCE TO RELATED APPLICATIONS

This application claims priority to U.S. Provisional Application No. 60/354,519 entitled “Biomarkers of Human Skin Cells” filed Feb. 8, 2002, which is hereby entirely and completely incorporated by reference.

### BACKGROUND OF THE INVENTION

1. Field of the Invention

The invention relates in general to biomarkers and, in particular, to differentially-expressed biomarker genes of mammalian skin-derived cells. The invention provides a plurality of up-regulated (signature) and down-regulated (anti-signature) genes for human keratinocytes, melanocytes, and fibroblasts, at normal and abnormal states such as non-cancerous and cancerous. The invention further provides analytical methods for identifying biomarker genes based on nucleic acid microarray data. The invention further relates to biomarkers of skin for use in molecular diagnostic and pathology applications in normal and abnormal tissues and cells.

2. Description of the Background

Cells of multicellular organisms, including mammalian species (e.g., humans), express characteristic biomarker genes that are significant in the corresponding cells' functions and distinguish between different cell and/or tissue types. Biomarkers not only enhance researcher's understanding of cell functions but also hold great promise as diagnostics for human disorders or pathologies involving abnormal cells. The identification of biomarker genes is an on-going pursuit of biomedical researchers.

Because of human skin tissue's complexity and the diverse cell types involved, identification of human skin cell biomarkers is of particular importance. Human skin is subject to a great many genetic and epigenetic disorders including, for example, cancer, psoriasis, and inflammatory conditions. Skin cell biomarkers will thus enable development of effective diagnostic products—and hence further aid in the discovery and characterization of therapeutics—for the skin disorders.

Keratinocytes, melanocytes, and fibroblasts are among the most abundant and important cell types of human skin. Keratinocytes are the most abundant cell type and reside in the epidermis where they form cornified layers that help to contain body fluids and provide barrier protection from the environment. Fartasch M and Ponec M, J. Invest. Dermatol. 102, 366-374. Keratinocytes are ectodermally derived and play essential roles in the formation of hair, nails and sebum. Fuchs E., Harvey Lect. 94, 47-77. Melanocytes are derived from the neural crest and are located in the lower epidermis and hair follicles where they generate melanin to provide coloration and protection from solar ultraviolet (UV) damage. Sturm R A et al., Bioessays 20, 712-721. Fibroblasts in the underlying dermis are derived from mesenchyme and synthesize essential extracellular matrix (ECM) components to provide structural support and elasticity. Takeda K et al., J. Cell Physiol. 153, 450-459. Certain genes may be uniquely expressed at a higher level in one of these cell types (i.e., up-regulated “signature” genes), while certain other genes may be uniquely expressed at a lower lever (or absent) in one of these cell types (i.e., down-regulated “anti-signature” genes). Both the over- and under-expressed genes can have diagnostic value, and can be useful in prognosis of disease severity and patient outcome.

Over the years, numerous gene products (and their mRNA transcripts) have been identified and reported as biomarkers of specific cell types of human skin. Usually these proteins and mRNAs have been discovered and studied one or a few at a time. In recent years, the evolution of nucleic acid microarray technologies has enabled researchers to simultaneously monitor expression patterns of thousands of genes, using oligonucleotide and DNA probes designed and/or selected based on the newly available genomic or cDNA sequence information. See, e.g., Zhao N. et al., Gene 156: 207-213; Schena M. et al., Science 270:467-470; Cole J. et al., Wound Repair Regen 9: 77-85. Although such microarray expression experiments have provided useful results, they are generally expensive to perform and often difficult to interpret.

The DNA microarray system DermArray® is useful for gene expression surveys in dermatology and related research and for selecting “highly-informative” genes for inclusion in nucleic acid microarrays (PCT/US01/01250 and U.S. patent application Ser. No. 09/759,377). With DermArray® one can screen thousands of genes for their expression levels in skin cells such as keratinocytes, melanocytes, and fibroblasts. DermArray® microarrays contain sequence-validated human cDNAs of genes for which some function is known as well as genes of unknown function (i.e., expressed sequence tags, ESTs).

There is a need for effective bioinformatic methods to analyze nucleic acid microarray data. In addition, there is a need to use said bioinformatic methods to identify new biomarkers for each of the cell types of mammalian (e.g., human) skin.

### SUMMARY OF THE INVENTION

The present invention overcomes the problems and disadvantages associated with current strategies and designs, and provides new biomarkers and methods for the detection and analysis of cell types and, in particular, mammalian skin.

One embodiment of the invention is directed to methods for identifying one or more biomarker genes for a first type of cells among a group of m different types of cells, from a multiplicity of genes whose expression levels in cells of the group are measured using one or more nucleic acid (or nucleotide) arrays, thereby generating a plurality of measurements of expression levels for the m types of cells, which method comprises: (a) calculating, for each gene, a “likelihood ratio” in the first type of cells by dividing (i) the product of (m−1) and the measurement for the first type of cells by (ii) the sum of the measurements for the m types of cells excluding the measurement for the first type of cells; (b) repeating step (a) for (m−1) times to calculate, for the each gene, a likelihood ratio in each of the m types of cells excluding the first type of cells, thereby obtaining (m−1) likelihood ratios for the gene; and (c) comparing the likelihood ratio of step (a) with the (m−1) likelihood ratios of step (b) for each gene and thereby determining a rank order for the each gene among the multiplicity, wherein the one or more biomarker genes are identified from the rank order.

According to the invention, a natural logarithm is taken for each likelihood ratio calculated for each gene in each type of cells in the group and the natural logarithm is designated as the “Gibbs likelihood” for each gene, wherein the rank order is determined according to the Gibbs likelihood for each gene among the multiplicity.

According to the invention, ordering may be performed for each gene by the Gibbs likelihoods, or sum of the Gibbs likelihoods for said gene in the m types of cells, or average of the Gibbs likelihoods for said gene in the m types of cells, thereby generating a Gibbs gene expression rank, wherein the rank order is determined based on the Gibbs gene expression rank.

According to the invention, an arithmetic mean of the Gibbs likelihoods for the genes in the multiplicity is taken and a standard deviation of the Gibbs likelihoods in the m types of cells is assessed, wherein the Gibbs likelihoods for the each gene in the first type of cells is represented in the units of the standard deviation plus or minus the corresponding arithmetic mean thereby determining a rank for the each gene in the rank order.

According to the invention, one or more genes with a Gibbs likelihood greater than u times the standard deviation are designated as signature biomarker genes of the first type of cells. In another embodiment, u is greater than 1, preferably equals 2.

According to the invention, one or more genes with a Gibbs likelihood ratio smaller than v times the standard deviation are designated as anti-signature biomarker genes of the first type of cells. In another embodiment, v is greater than 1, preferably equals 2.

According to the invention, a median is taken for the likelihood ratios calculated for each gene in the m types of cells, the median being designated as the “median likelihood”, wherein the rank order is determined according to the median likelihood for each gene among the multiplicity.

According to the invention, comparing further comprises generating a median rank distribution by sorting the genes in the multiplicity according to the corresponding median likelihoods, wherein the rank order is determined based on the median gene expression rank.

According to the invention, an arithmetic mean of the median likelihoods for the genes in the multiplicity is taken and a standard deviation of the median likelihoods in the m types of cells is assessed, wherein the median likelihoods for the each gene in the first type of cells is represented in the units of the standard deviation plus or minus the corresponding arithmetic mean thereby determining a rank for the each gene in the rank order.

According to the invention, one or more genes with a median likelihood greater than u times the standard deviation are designated as anti-signature biomarker genes of the first type of cells. In another embodiment, u is greater than one, preferably equals two.

According to the invention, one or more genes with a median likelihood ratio smaller than v times the standard deviation are designated as signature biomarker genes of the first type of cells. In another embodiment, v is greater than one, preferably equals two. m is greater than or equals three. The different types of biological samples for evaluation may be cells or tissues that are normal or abnormal. The different types of cells are preferably skin cells and skin cells may comprise keratinocytes, melanocytes, and fibroblasts. In another embodiment, the skin cells comprise normal melanocytes, cutaneous primary melanoma cells, and metastatic melanoma cells. In another embodiment, the skin cells are derived from a mammal (e.g., human).

According to the invention, the gene may be selected from the group comprising transducer of ERBB2 member 2, Finkel-Biskis-Reilly murine sarcoma virus, RAB6, KIAA0996 protein, homeo box A10, Tax1 binding protein 1, SET binding factor 1, ubiquitination factor E4A, solute carrier family 1 member 3, heterogeneous nuclear ribonucleoprotein A3, EST cDNA ID 471826, EST cDNA ID 206907, EST cDNA ID 427657, and EST cDNA ID 208082 as set forth in Table 10 which gene is used as a signature (up-regulated) biomarker of metastatic melanoma cells.

The gene may also be selected from the group comprising histidyl-tRNA synthetase homolog and an EST cDNA ID 209841 as set forth in Table 9, which gene is used as a signature (up-regulated) biomarker of cutaneous primary melanoma cells.

The gene may also be selected from the group comprising nidogen 2, erythroid alpha-spectrin 1, afx1 transcription factor, and sarcoma-amplified sequence, which gene is used as a signature (up-regulated) biomarker of normal melanocytes (when compared to melanoma cells).

The gene may also be selected from the group comprising fibroblast growth factor 12, intercellular adhesion molecule 2, hematopoietic protein 1, interleukin-1 receptor-associated kinase, and CD163, which gene is used as an anti-signature (down-regulated) biomarker for metastatic melanoma cells.

The gene may also be selected from the group comprising small proline-rich protein 2D, type VIII collagen alpha 1, trophinin, chondroitin sulfate proteoglycan 3, type IV collagen alpha 4, activin A receptor type II-like 1, paired box gene 6, homeobox D4, homeobox B5, zinc finger protein 131, special AT-rich sequence binding 1, ubiquitin specific protease 16, pyrolin-5-carboxylate synthetase, neural expressed developmentally down-regulated 5, ribonuclease P (30 kD), protein tyrosine phosphatase (rec F), endothelial lipase, ras homolog gene, valyl-tRNA synthetase 2, arylsulfatase A, aldo-keto reductase 1C1, protein phosphatase 1 (regulatory 3C), development regulated GTP-binding 1,3-hydroxybutyrate dehydrogenase, adipose most abundant transcript, pancreatic polypeptide 2, solute carrier 11A2, cardiac ankyrin repeat protein, heparin-binding GF binding protein, Ewing sarcoma break point region 1, and DHHC1 protein, which gene is used as a signature (up-regulated) biomarker for keratinocytes.

The gene may also be selected from the group comprising EGF-related fibulin 5, gamma interleukin 2 receptor, eukaryotic translation elongation factor 2, mitochondrial ribosomal protein L23, ribosomal protein L7a, SEC23-like protein B, solute carrier family 16A3, metallothionein 1F, metallothionein 1H, interferon induced transmembrane 2, Dickkopf homolog 3, mucin-related episialin, high mobility group protein I-C, and growth factor receptor-bound protein 14, which gene is used as a signature (up-regulated) biomarker for fibroblasts.

The gene may also be selected from the group comprising galectin 3, syndecan binding protein (syntenin), dystroglycan 1, prostate differentiation factor, glutaminyl cyclotransferase, Na+/K+ transporting ATPase alpha 1, cAMP-dependent protein kinase I alpha 1, protein tyrosine phosphatase IVA 2, fyn oncogene, 6-pyruvoyl-tetrahydropterin synthase, dihydopyrimidinase, pirin, major histocompatibility complex I-C, 4F2 antigen heavy chain (solute carrier 3), abl-interactor 2b, coxsackie virus and adenovirus receptor, prostatic binding protein, proteolipid protein 1, v-abl 1, ets2 repressor factor, proline-rich Gla 1, axin 1 up-regulated, voltage-gated K+ channel beta subunit, EST cDNA ID 712604, EST cDNA ID 267859, EST cDNA ID 320588, EST cDNA ID 1048698, EST cDNA ID 305843, as set forth in Table 3, which gene is used as a signature (up-regulated) biomarker of melanocytes.

The gene may also be selected from the group comprising microtubule-associated protein 1B, monocytic leukemia zinc finger protein, Clathrin heavy chain 1, non-metastatic cells 4, TC10-like Rho GTPase, Myelin gene expression factor 2, and CAAX box 1, which gene is used as an anti-signature (down-regulated) biomarker for keratinocytes.

The gene may also be selected from the group comprising long chain 2 of Fatty-acid coenzyme A ligase, calcium modulating ligand, and nuclear receptor coactivator 3, which gene is used as an anti-signature (down-regulated) biomarker for fibroblasts.

The gene may also be selected from the group comprising ribosomal protein L30 and orosomucoid 1, which gene is used as an anti-signature (down-regulated) biomarker for melanocytes.

Another embodiment of the invention is directed to bioinformatic methods for analyzing gene expression data generated from nucleic acid microarray experiments to identify further biomarker genes from various cell types.

Another embodiment of the invention is directed to biomarker genes identified from mammalian (e.g., human, primate) keratinocytes, melanocytes, and fibroblasts, at normal and/or abnormal states. The biomarker genes are useful as molecular targets for therapeutics of a disorder or disease in mammals.

Other objects and advantages of the invention are set forth, in part, in the description, which follows, and in part, will be obvious from this description and may be learned from the practice of the invention.

### DESCRIPTION OF TABLES AND DRAWINGS

With regard to the Tables, the column “Function” contains general descriptions of the corresponding gene function. The column “cDNA ID” contains the clone designation numbers in the I.M.A.G.E. Consortium, of the Lawrence Livermore National Laboratory (listed sequences can be identified at http://image.llnl.gov and/or http://ncbi.nim.nih.gov). The column “Gene” contains common names of the genes. “Symbol” contains standard symbols for the gene products. Where appropriate, the columns “K,” “F,” and “M” list likelihood ratios calculated for the samples from keratinocytes, fibroblasts, and melanocytes, respectively, and the columns “N,” “P,” and “M” list Gibbs likelihoods calculated for the samples from normal melanocytes (NHEM), primary cutaneous melanoma (MS7), and metastatic melanoma (SKMel-28), respectively. In addition, the simple intensity ratios for each gene are shown in the columns “P/N” and “P/M.” Simple ratios indicating a more than two-fold (or the inverse) change are emboldened. The column “Reference” lists the relevant reference articles, if known, relating to the corresponding genes, including first author and year of publication, and obtained via PubMed literature searches online.

Table 1 shows a list of keratinocyte signature (up-regulated) biomarkers identified according to this invention (and when the comparison group consisted of RNA samples from keratinocytes, fibroblasts, and melanocytes).

Table 2 shows a list of fibroblast signature (up-regulated) biomarkers identified according to this invention (and when the comparison group consisted of RNA samples from keratinocytes, fibroblasts, and melanocytes).

Table 3 shows a list of melanocyte signature (up-regulated) biomarkers identified according to this invention (and when the comparison group consisted of RNA samples from keratinocytes, fibroblasts, and melanocytes).

Table 4 shows a list of keratinocyte anti-signature (down-regulated) biomarkers identified according to this invention (and when the comparison group consisted of RNA samples from keratinocytes, fibroblasts, and melanocytes).

Table 5 shows a list of fibroblast anti-signature (down-regulated) biomarkers identified according to this invention (and when the comparison group consisted of RNA samples from keratinocytes, fibroblasts, and melanocytes).

Table 6 shows a list of melanocyte anti-signature (down-regulated) biomarkers identified according to this invention (and when the comparison group consisted of RNA samples from keratinocytes, fibroblasts, and melanocytes).

Table 7 shows the primers used for the qRT-PCR experiments for verifying results of nine signature (up-regulated) biomarker genes from DNA microarray studies using DermArray® and RNA samples from keratinocytes, fibroblasts, and melanocytes. The keratinocyte biomarkers include keratins 5, 14, and 19 (KRT 5, 14, and 19 respectively). The fibroblast biomarkers include apolipoprotein D, collagen 6 A1, vimentin (APOD, COL6A, and VIM, respectively). The melanocyte biomarkers include melan-A, silver, and tyrosinase-related protein 1 (MLANA, SILV, and TRP1, respectively).

Table 8 shows the results of the qRT-PCR experiments (using the PCR primers from Table 7) for verifying results from microarray studies using DermArray®. Three RNA samples were used: keratinocytes, K; dermal fibroblasts, F; and melanocytes, M. DermArray® hybridization intensities (I_{K}, I_{F}, and I_{M}) were measured for nine signature (up-regulated) biomarker genes. DermArray® likelihood ratios, L_{K}, L_{F}, and L_{M,} were calculated from the intensities and compared to qRT-PCR results, expressed as yields of double stranded DNA in nanograms [ng].

Table 9 shows a list of MS7 primary cutaneous melanoma cell line biomarkers identified according to this invention (and when the comparison group consisted of RNA samples from cultured normal melanocytes, MS7 primary cutaneous melanoma cell line, and SKMel-28 metastatic melanoma cell line). The top panel includes the signature genes while the bottom panel include the anti-signature genes.

Table 10 shows a list of the SKMel-28 metastatic melanoma biomarkers identified according to this invention (and when the comparison group consisted of RNA samples from cultured normal melanocytes, MS7 primary cutaneous melanoma cell line, and SKMel-28 metastatic melanoma cell line). The top panel includes the signature genes while the bottom panel include the anti-signature genes.

Table 11 shows a list of normal melanocytes biomarkers identified according to this invention (and when the comparison group consisted of RNA samples from cultured normal melanocytes, MS7 primary cutaneous melanoma cell line, and SKMel-28 metastatic melanoma cell line). The top panel include the signature genes while the bottom panel include the anti-signature genes.

FIG. 1 shows scatter plots of DermArray® hybridization intensities on logarithmic scales for keratinocytes (K), melanocytes (M), and dermal fibroblasts (F) according to one embodiment of this invention (and when the comparison group consisted of RNA samples from keratinocytes, fibroblasts, and melanocytes). Each of the 4,405 human genes detected on the array is represented as a single dot. Data points that fall outside of the diagonal indicate potential biomarker genes. The top panel is the scatter plot of M vs. K; the middle panel is the scatter plot of F vs. K; and the bottom panel is the scatter plot of M vs. F.

FIG. 2 shows the distribution of Gibbs likelihood values for the 4,405 human genes with regard to keratinocytes (K) detected on the DNA microarray according to one embodiment of this invention (and when the comparison group consisted of RNA samples from keratinocytes, fibroblasts, and melanocytes) plotted against the Gibbs ranking index, as displayed in standard deviation (SD) units. The data points outside of a SD range of −2 to +2 may be considered as potential biomarker genes. The inset figure highlights the symmetry of the bell-shaped distribution.

FIG. 3 shows a scatter plot of Gibbs likelihood values according to one embodiment of this invention (and when the comparison group consisted of RNA samples from keratinocytes, fibroblasts, and melanocytes). The top panel is a scatter plot of Gibbs likelihood values of M vs. F (with K as internal reference), as displayed in standard deviation (SD) units; and, the bottom panel is a schematic depicting the result of the top panel scatter plot. Data points that are outside of the circle with a radius of two implicate potential signature and/or anti-signature marker genes: Particularly, according to one embodiment of this invention, those which fall in quadrant IV (upper left) may be considered as melanocyte signature genes; those which fall in quadrant III (lower left) may be considered as keratinocyte signature genes; those which fall in quadrant II (lower right) may be considered as fibroblast signature genes; and those fall in quadrant I (upper right) may be considered as keratinocyte anti-signature genes.

FIG. 4 shows, on the left panel, the Median likelihoods plotted against the median likelihood ranking index calculated from the triplicated genes on DermArray® microarrays for one of the cell types according to one embodiment of this invention (and when the comparison group consisted of RNA samples from keratinocytes, fibroblasts, and melanocytes), the likelihoods being displayed in standard deviation (SD) units. The positive and negative cutoff ratios were defined as equal to the mean plus or minus twice the standard deviation, respectively, in one embodiment. The data points above the positive cutoff ratio were considered as anti-signature genes whereas those below the negative cutoff ratio were considered as signature genes. On the right panel, three schematics demonstrate the criteria for determining signature, anti-signature, and variable genes (and comparing a minimum of three RNA samples) according to one embodiment of this invention.

### DESCRIPTION OF THE INVENTION

Description of Relevant Terms

As used herein, the term “microarray” refers to nucleic acid or nucleotide arrays or protein or peptide arrays that can be used to detect biomolecules, for instance to measure gene expression. “Array,” “microarray”, “nylon filter”, “slide,” and “chip” are used interchangeably in this disclosure. Various kinds of arrays are made in research and manufacturing facilities worldwide, some of which are available commercially. There are, for example, two main kinds of nucleic acid arrays that differ in the manner in which the nucleic acid materials are placed onto the array substrate: spotted arrays and in situ synthesized arrays. One of the most widely used oligonucleotide arrays is GeneChip™ made by Affymetrix, Inc. The oligonucleotide probes that are 20- or 25-base long are synthesized in silico on the array substrate. These arrays tend to achieve high densities (e.g., more than 40,000 genes per cm²). The spotted arrays, on the other hand, tend to have lower densities, but the probes, typically partial cDNA molecules, usually are much longer than 20- or 25-mers. Representative types of spotted cDNA arrays include LifeArray made by Incyte Genomics and DermArray made by IntegriDerm (or Invitrogen). Pre-synthesized and amplified cDNA sequences are attached to the substrate of these kinds of arrays. Protein and peptide arrays also are known. See Zhu et al., Science 293:2101 (2001).

Particularly, in one embodiment of this invention, DermArray® was used. DermArray® DNA microarrays (ID1001 by IntegriDerm Inc.) were created by empirical survey of gene expression in skin-derived cells using a panel of GeneFilters® DNA microarrays (ResGen/Invitrogen, see www.invitrogen.com) which at that time contained approximately 26,000 unique, sequence validated human cDNAs. Proprietary methods were used to select genes that were differentially expressed in keratinocytes, fibroblasts, and melanocytes for inclusion on the DermArray® filters. See, U.S. application Ser. No. 09/759, 377. The list of genes includes ca. 4405 unique cDNAs, with 4025 empirically chosen and 383 whose importance in dermatology have been well established in the literature. The 4025 cDNAs are spotted once on the array whereas the 383 cDNAs are spotted in triplicate. See either www.integriderm.com or www.dermarray.com for additional information.

Microarray data, as used herein, encompasses any data generated using various arrays, including but not limited to the nucleic acid arrays described above. Typical microarray data include collections of gene expression levels measured using nucleic acid arrays on biological samples of different biological states and origins. The methods of the present invention may be employed to analyze any microarray data; irrespective of the particular nucleic acid microarray platform (e.g., nylon filters, glass slides, plastic, or silicon chips) from which the data are generated.

Gene expression, as used herein, refers in general to the transcription from DNA sequences into RNA molecules, which encode certain proteins with structural, enzymatic, or regulatory functions. The expression level of a given gene measured at the nucleotide level refers to the amount of RNA transcribed from the gene measured on a relevant or absolute quantitative scale, and in general refers to the relative abundance of the accumulated mRNA transcript. The expression level of a given gene measured at the protein level refers to the amount of protein translated from the transcribed RNA measured on a relevant or absolute quantitative scale. The measurement can be, for example, an optical density value of a fluorescent or radioactive signal, on a blot or a microarray image. Differential expression, as used herein, means that the expression levels of certain genes, as measured at the RNA or protein level, are different between biological samples in different states, tissues, or type of cells. Differential expression may also be observed relative to a reference standard. Such standard may be determined based on the context of the expression experiments, the biological properties of the genes under study, and/or statistical significance criteria.

Simple ratio, as used herein, refers to, with respect to a gene, is the ratio of its hybridization intensity measured from a first sample or a first group of samples to its hybridization intensity measured from a second sample or a second group of samples. The first and second samples or groups of samples may be from different tissues, types of cells; or they may correlate with different biological and/or pathological states, according to various embodiments of this invention. The hybridization intensities may be normalized before the ratio is calculated according to certain embodiments, to account for the background noise, the bias introduced by the different samples, among other things.

Likelihood ratio, as used herein, refers to, with respect to a gene, the ratio of its hybridization intensity measured from a first sample or a first group of samples to the mean of its hybridization intensities measured from all the other samples or groups of samples in a given experiment. These samples or groups of samples may be obtained from different tissues, types of cells; or they may correlate with different biological and/or pathological states, according to various embodiments of this invention. Thus, likelihood ratios reflect the likelihood that a gene is expressed in one tissue, cell type, or at a particular biological state vis-à-vis other cell types, tissues, or biological states. In various embodiments, likelihood ratios for an experiment involving three cell types, including keratinocytes (K), melanocytes (M), and fibroblasts (F) may be calculated as follows:

$$\begin{matrix}{R}_{K} = \frac{2 \cdot {I}_{K}}{( {I}_{F} + {I}_{M} )} & {R}_{M} = \frac{2 \cdot {I}_{M}}{( {I}_{F} + {I}_{K} )} & {R}_{F} =\end{matrix} \frac{2 \cdot {I}_{F}}{( {I}_{K} + {I}_{M} )}$$

where Rₖ, Rₘ, and R_{f} represent likelihood ratios for the three cell types and Iₖ, Iₘ, and I_{f} represent hybridization intensities for each of the cell types in the DNA microarray experiment.

Median likelihood refers to, with respect to a gene, the median value of its likelihood ratios for all the cell types or tissues considered in an experiment. That is, Median Likelihood_{Gene x}=median (R_{N}, R_{P}, R_{M}).

Gibbs likelihood refers to, with respect to a gene, the natural logarithms of the likelihood ratio. The sum of the Gibbs likelihood values for each gene may also be calculated to serve in ranking or ordering all of the genes within a biological sample or for all the biological samples (cell types or tissues) considered in an experiment. The name was assigned by analogy to Gibbs free energy calculations in other scientific contexts. That is, Gibbs Likelihood _{Gene x}=ln R_{N}+ln R_{P}+ln R_{M}

Signature gene, as used herein, refers to a biomarker gene whose expression is significantly up regulated in one cell type or tissue compared to other cell types or tissues, and in the embodiments provided is determined by likelihood ratios (or simple ratios). That is, for example, the gene's likelihood ratio (or simple ratio) is significantly higher in one cell type or tissue (hence up-regulated therein) than in all other cell types or tissues considered in an experiment. The significant level may be empirically designated, or determined by any suitable statistical standard, or assigned arbitrarily.

Anti-signature gene, as used herein, refers to a biomarker gene whose expression is significantly down regulated in one cell type or tissue compared to other cell types or tissues, and in the embodiments provided is determined by likelihood ratios (or simple ratios). That is, for example, the gene's likelihood ratio (or simple ratio) is significantly lower in one cell type or tissue (hence down-regulated therein) than in all other cell types or tissues considered in an experiment. The significant level may be empirically designated, or determined by any suitable statistical standard, or assigned arbitrarily.

Variable Gene, as used herein, refers to a gene that is not signature or anti-signature gene of a particular cell type or tissue. That is, it may be up regulated in one or more cell types or tissues, down regulated in one or more cell types or tissues, or expressed at intermediate ranges in one or more cell types or tissues.

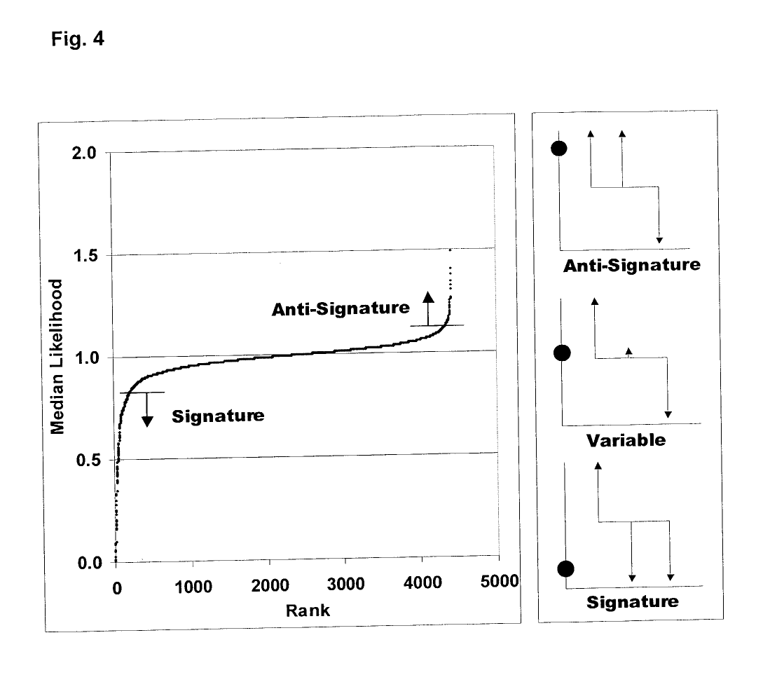

Gene expression rank, as used herein, refers to two kinds of ranks, the first is based on the median likelihoods and the second is based on the Gibbs likelihoods. In the first case, genes are rank ordered by the median likelihoods. Genes that are more likely up-regulated in one specific cell type or tissue (hence signature genes thereof) have low median values and accordingly are ranked low, as reflected in FIG. 4. Genes that are more likely down-regulated in one specific cell type or tissue (hence anti-signature genes thereof) have high median values and accordingly ranked high, also reflected in FIG. 4. According to one embodiment of this invention, using the median likelihood rank, genes with ranks greater than average plus twice the standard deviation are designated as anti-signature genes, and genes with rank less than average minus twice the standard deviation are designated as signature genes.

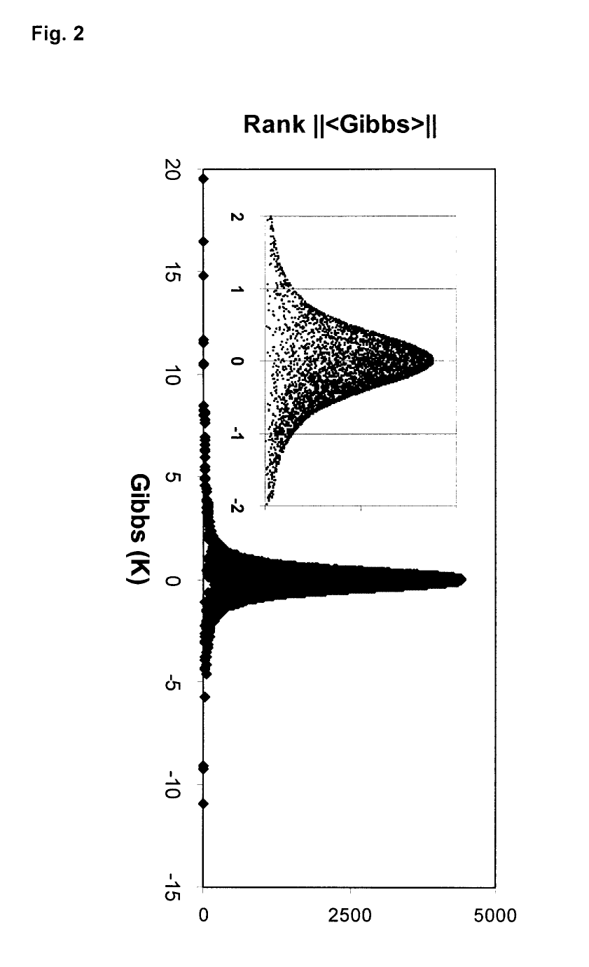

In the second case, genes are rank ordered by the Gibbs likelihoods for all the cell types or tissues. Gene expression distribution, as used herein, refers to a distribution of Gibbs likelihood for a particular cell type or tissue plotted over Gibbs likelihood rank of all the genes. As shown in FIG. 2, when these curves are plotted in standard deviation units, signature or anti-signature genes may be identified by visualization: The genes towards the tails at both directions are the significantly up- or down-regulated in a particular cell type or tissue and hence represent signature or anti-signature genes thereof, respectively.

Identifying Biomarker Genes Based On Likelihood Ratios

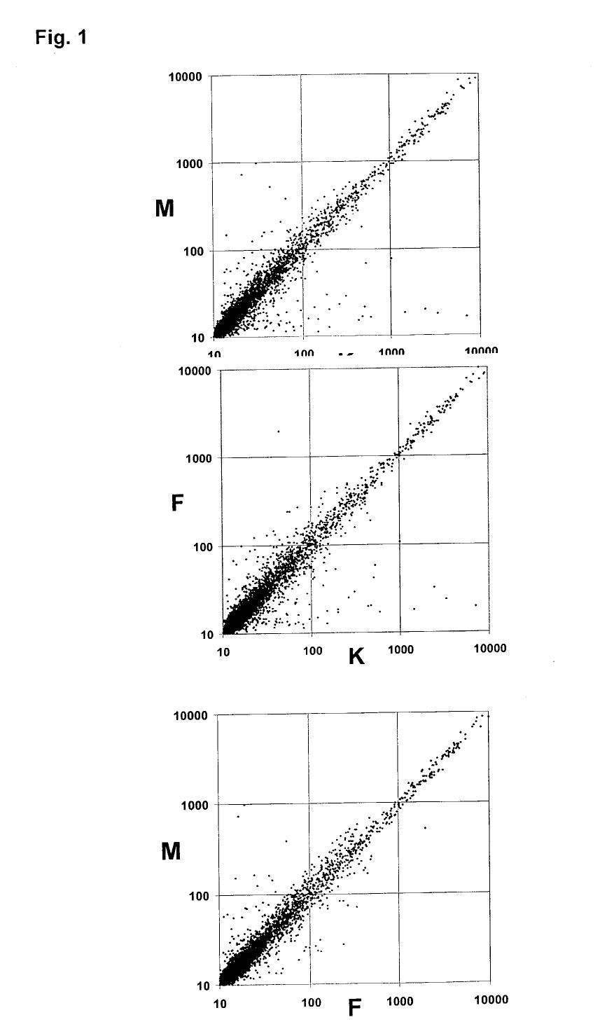

Microarray expression studies may be performed using biological samples from different tissues, cell lines, or different biological or pathological states. The resultant hybridization intensity data can then be analyzed to identify potential biomarker signature and anti-signature genes for the corresponding cells at different states. In one embodiment, raw intensity data from DermArray® hybridization experiments using keratinocyte-, fibroblast- and melanocyte-derived radiolabeled probes may be obtained and processed using Pathways 2 software (Invitrogen—ResGen). Intensities may be normalized and corrected for background signals. FIG. 1 shows the scatter plots of the normalized intensities obtained from such a DermArray® experiment. Different pairs of comparisons are shown: melanocyte-keratinocyte in the upper panel, fibroblast-keratinocyte in the middle panel, and melanocyte-fibroblast in the lower panel. Each data point may represent one gene or the mean of multiple replicate measurements (e.g., triplicates) of one gene in various embodiments. Data points that lie along the diagonal of these scatter plots represent genes expressed at comparable (approximately invariant) levels in both cell types, whereas data points that lie off diagonal represent genes expressed at greater levels in the cell type designated by the nearer axis. Thus, hundreds of genes are shown to be differentially expressed in the three cell types; and, keratinocytes demonstrate more over-expressed genes than melanocytes or fibroblasts using DermArray®.

As described herein, a likelihood ratio represents the likelihood or probability of a gene being expressed in one cell (or tissue) type compared to other cell (or tissue) types in a group. A group may include three or more cell (or tissue) types according to this invention. Applying Gibbs likelihoods, a gene expression rank may be established for a group of cell (or tissue) types by sorting genes by their Gibbs likelihoods. Referring to FIG. 2, the Gibbs likelihoods for the keratinocyte distribution are plotted (in standard deviation units) vertically against the gene expression rank horizontally, resulting in a bell-shaped distribution. The distribution is centered on zero for genes that express equally in all three samples. In certain embodiments, data points above two (+2) and below negative two (−2) are designated as representing signature or anti-signature genes, respectively.

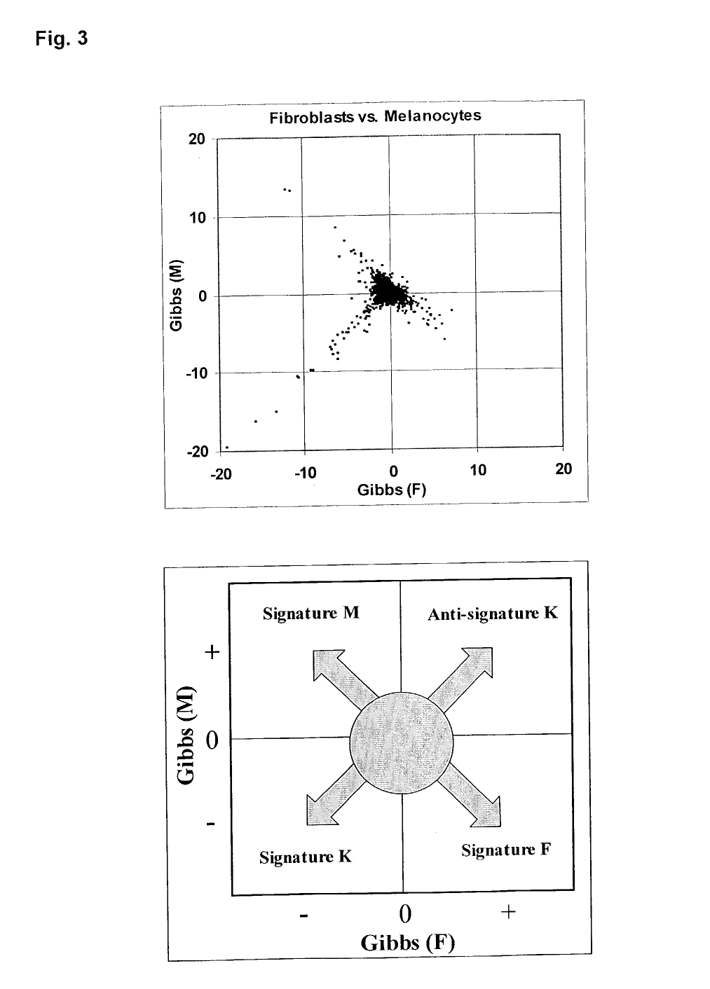

Referring to FIG. 3, the upper panel, Gibbs likelihoods for the three cell types are shown in a scatter plot. The data is expressed in units of standard deviations of the Gibbs likelihoods. In certain embodiments, genes represented by the data points outside of the sphere of radius two in the Cartesian plane are designated as the signature (up-regulated) or anti-signature (down-regulated) biomarker genes for the corresponding cell types. The results shown in the upper panel of FIG. 3 is illustrated further in the lower panel of FIG. 3. The anti-correlated data points represent either fibroblast signature genes (quadrant II) or melanocyte signature genes (quadrant IV). Downward-correlated data points (quadrant III) represent keratinocyte signature genes; and, the upwards-correlated data points (quadrant I) represent keratinocyte anti-signature genes.

The Gibbs likelihood method for identifying biomarker genes according to this invention is capable of identifying potential signature, anti-signature, as well as variable genes. The variable genes are less obvious biomarkers. In an alternative embodiment, median likelihood ratios are used to identify biomarker genes. This median likelihood method removes the variable genes; it only selects potential signature and anti-signature genes.

For example, referring to FIG. 4, genes from three hybridization experiments using the different types of cells are rank ordered according to the median likelihood ratios. The genes with median likelihood ratio less than the mean (0.9775) minus two times the standard deviation of this index (0.1036) are categorized as signature genes (median<0.7649). And, the genes with the median likelihood ratios greater than the mean plus two times the standard deviation of the index are categorized as anti-signature genes (median>1.1902). Different threshold numbers (e.g., one time or more than two times standard deviation units) or other suitable statistical standards may be adopted in other embodiments according to this invention to designate signature and anti-signature genes for various cell types and based on the specific microarray data obtained.

Tables 1-3 show a list of up-regulated genes—hence signature genes—identified using the aforementioned methods in normal human skin keratinocytes, fibroblasts, and melanocytes, respectively. A total of 136 signature biomarker genes are identified; 66 in keratinocytes, 32 in fibroblasts, and 38 in melanocytes. The genes are displayed in descending order according to their likelihood ratios in the corresponding cell type, and grouped by similar functions (e.g., enzymes, cytokines).

Tables 4-6 shows a list of down-regulated genes—hence anti-signature genes—identified using the aforementioned methods in normal human skin keratinocytes, fibroblasts, and melanocytes, respectively. Thirteen of these genes are keratinocyte biomarkers, four are melanocyte biomarkers, and five are fibroblast biomarkers. Thus, in the examples provided in Tables 1-6 there are less anti-signature genes identified than the signature genes for all the cell types. The difference in the numbers of identified signature and anti-signature genes might reflect a bias in the list of genes immobilized on the DermArray® filters.

Keratinocyte Signature Biomarker Genes

Intermediate filament proteins Keratin 5 and 14 are dimerization partners and well-established biomarkers of basal keratinocytes. Jiang C K. et al., Growth Factors 12, 87-97. Mutations in either of these genes cause a blistering disorder of human skin, epidermolysis bullosa simplex. See, Lane E B et al., Nature, 356:244-246; J. Invest. Dermatol. 105:629-632. Likelihood ratios for these two genes are approximately 200-400 fold higher in keratinocytes compared to fibroblasts or melanocytes, as shown in Table 1. Among the suprabasal keratins 1 and 10 (Poumay Y and Pittelkow M R, J. Invest. Dermatol. 104:271-276) and the wound-repair associated keratins 6 andl6 (Hutton E. et al., J. Cell Biol., 143:487-499) on DermArray®, only 6B is identified as a strong keratinocyte signature biomarker, as shown in Table 1. It is conceivable that keratins 1 and 10 might be detected to be up-to regulated under the differentiation-inducing conditions.

Keratins 4 and 13 are dimerization partners and recognized biomarkers of stratified non-comified mucosal keratinocyte cells. McGowan K and Coulombe P A, Subcel. Biochem. 31:173-204. Keratin 13 is a strong keratinocyte signature biomarker identified by the method of this invention. But the likelihood ratio of keratin 4 is moderate in keratinocytes. Keratins 7, 8, 18, and 19 are well-known biomarkers of simple epithelial cells. Hutton et al. J. Cell Biol. 143:487-499. They are all identified as signature biomarkers of keratinocytes by the method of this invention, as shown in Table 1. In particular, keratin 19 is a predictor of rapid cell growth and is considered to be a biomarker of keratinocyte stem cells. Lu M H, et al., Proc. Natl. Sci. Counc. Repub. China B. 24:169-177.

A number of genes that are associated with extracellular matrix (ECM) and adhesion of keratinocytes are identified by the method of this invention to be signature biomarkers of these cells. Desmoplakin plays a key role in adhesion. Gallicano G I et al., J. Cell Biol., 143:2009-2022. Collagens 4 and 7 are well-characterized structural anchors of keratinocytes in skin, located under the basement membrane. Wille M S and Furcht L T, J. Invest. Dermatol. 95:264-270. Using the likelihood ratio method of this invention, both collagens show signature expression in the keratinocytes (Col7a is just below the level of significance). Collagen 8 appears to be another signature biomarker of keratinocytes, identified by the likelihood ratio method of this invention.

A cluster of keratinocyte-specific genes known as the epidermal differentiation complex (EDC) has been localized to chromosome 1q21. Marenholz I et al., Genomics 37:295-302. This complex includes the structural proteins loricrin (not on the DermArray® filters), involucrin, the small proline rich proteins, trichohyalin, profilaggrin, and cornifin, which are expressed during cornification, and approximately a dozen members of the S100 family (annexins). Mischke D. et al., J. Invest. Dermatol. 106:989-992. Annexins bind calcium, which exerts a pro-differentiation effect on keratinocytes in vitro. Ma A S and Ozers L J. Arch. Dermatol. Res. 288:596-603.

Four or five of the annexins (A2, A8 and A9, A10 and A11) are identified to be signature biomarkers of keratinocytes. Most notably, S100A2 has a high likelihood ratio, and is a well know tumor suppressor that is under-expressed in squamous cell carcinoma. Nagy N. Lab Invest. 81:599-612. It is also down-regulated in melanoma, and not expressed at all in metastatic melanoma. Boni R. et al. Br. J. Dermatol. 137:39-43. The presence of A2 indicates a positive prognosis for both diseases. Lauriola L et al., Int. J. Cancer 89: 345-349. The identification of A2 as a strong signature gene for normal keratinocytes is consistent and verifies those observations. The A8 and A9 proteins are generally associated as a pair and involved in injury response, inflammation, and tumor suppression. Thorey I S et al., J. Biol. Chem. 276: 35818-35825. Both are identified as signature markers of keratinocytes. The A10 and A11 genes are well-known substrates of transglutaminases; they are identified as signature genes of keratinocytes. A7 is also a substrate for tranglutaminases, however, it is not identified as a signature biomarker of any of the three cell types.

Two other genes associated with the EDC are identified to be signature genes of the keratinocytes: the small proline rich proteins SPRR1B and SPRR2C (cornifin). Cornifin is a well-known biomarker of cornification. Cabral A. et al. J. Biol. Chem. 276: 19231-19237. It has a high likelihood ratio and is identified as a signature biomarker of keratinocytes.

Homeobox proteins are transcription factors that regulate differentiation of many cell types including keratinocytes. Scott G A and Goldsmith L A., J. Invest. Dermatol. 101:3-8. Transcription of various homeobox genes up- or down-regulated at different stages of development, proliferation, and differentiation. Stelnicki E J et al., J. Invest. Dermatol., 110:110-115. The HOX subgroup of homeobox genes is localized in clusters A, B, C, and D on four different chromosomes. Each cluster contains 13 genes, for a total of 56 HOX genes. Magli M C et al. Proc. Natl. Acad. Sci. USA 88:6348-6352. Only HOXB5 and HOXD4 of the fourteen homeobox genes on the array are signature biomarkers of keratinocytes. Homeobox B5 (also known as HOX2A) is part of the HOXB gene cluster (also called the HOX2 cluster) localized at chromosome 17q21-q22 in the region of the type I (acidic) keratin genes. Lessin S R et al., J. Invest. Dermatol. 91:572-578. It is possible that HOX2A is involved in the regulation of the acidic keratins (i.e. keratin 14). However, three other HOXB genes on the array are not identified as signature markers of keratinocytes, suggesting that the association may be coincidental. The homeobox D4 gene (also known as HOX4B) is part of the HOXD gene cluster (also called HOX4). HOXD4 is localized on chromosome 2q31-q37 in the region of several collagens including the signature gene collagen 4A. Penkov et al., J. Biol. Chem. 275, 16681-16689. None of the three HOXA genes on the array (1, 5, or 10) is identified as signature biomarkers of any of the three cell types, even though HOXA genes have been associated with human skin development. Stelnicki E J. et al., J. Invest. Dermatol. 110:110-115.

Fibroblast Signature Biomarker Genes

Dermal fibroblasts synthesize connective tissues and compose the support matrix (stoma) of the dermis of skin. Fibroblasts are implicated in photoaging of skin. Hadshiew I M et al., Am. J. Contact Dermat. 11:19-25. Relative to young or normal skin, the dermis of photoaged skin has qualitative and quantitative differences in dermal collagen, elastins, and other structural components produced by fibroblasts. Yaar M and Gilchrest B A, J. Dermatol. Surg. Oncol. 16:915-922.

Referring to Table 2, an extracellular matrix (ECM), structural, and adhesion class of genes that includes vimentin, collagen 1A2, and 6A1, etc., are among the most discriminatory signature genes of normal fibroblasts, identified according to this invention. These genes are intimately associated with the extracellular matrix or the cytoskeleton. Geiger B et al. Nat. Rev. Mol. Cell. Biol. 2:793-805. Collagen 1A2 is a fibrillar forming collagen that is found in skin, bone tendon, and ligament. Mundlos S. et al. J. Biol. Chem. 271:21068-21074. Defects in this gene have been linked with defects in skin ranging from hyper-extendability to poor wound healing. Byers P H. Am J. Med. Genet. 34:72-80. Collagen 6 μl plays a critical role in cell-matrix adhesion to skeletal muscle. Lamande S R et al. Hum. Mol. Genet. 7:981-989. Vimentin is an intermediate filament phosphoprotein (Ferrar S. et al., Mol. Cell. Biol. 6: 3614-3620) that confers rigidity to circulating lymphocytes, and its collapse plays a role in transendothelial migration. Brown M J et al., J. Immunol. 166: 6640-6646. Some of the fibroblast biomarkers identified have previously been associated with cardiac tissue and endothelium.

Melanocyte Signature Biomarker Genes

Melanocytes are derived from neural crest cells during embryonic development. Pigmentation-related genes can serve as good signature biomarkers of the melanocyte cells. Jackson I J., Hum. Mol. Genet. 6:1613-1624; Hearing V J and Jimenez M, Pigment Cell Res., 2:75-85. Referring to Table 3, the method of this invention identifies a number of such genes, including, in descending order of median likelihood ratios, silver (SILV), melan A (MLANA), tyrosinase (TYR), ocular albinism 1 (OAC1), tyrosinase-related protein 2 (TYR), and tyrosinase-related protein 1 (TYP2). Silver and melan-A are robust signature biomarkers in melanocytes.

However, a number of other well-known pigmentation-related genes are not identified by their median likelihood ratios to be signature biomarkers of this cell type, such as microphthalmia associated transcription factor (MITF), agouti-signaling protein, proopiomelanocortin (ASIP), and melanocortin 1 receptor (MC1R). It is possible that these mRNAs are present in relatively low abundance to be detected, or the stringent bioinformatic selection critieria excluded them.

The method of this invention also identifies other melanocyte biomarkers besides the well-known pigmentation genes: One of the signaling proteins, glutaminyl-peptide cyclotransferase (QPCT), is a well-studied pituitary enzyme. Fischer W H and Spiess J. Proc. Natl. Acad. Sci. USA., 84:3628-3632. Glutaminyl cyclase is ten times more likely expressed in melanocytes than the other cultures. The major histocompatibility complex I gene (HLA-C) is four times more likely expressed in the melanocytes. Class I MHC genes are important in self vs. non-self recognition by the immune system. Natarajan K et al. Rev. Immunogenet. 1:32-46. They are expressed in most somatic cells, but are not usually expressed in the central nervous system. Moseley R P et al, J. Pathol., 181: 419-425.

Many annexin genes are up regulated in keratinocytes when detected by DermArray® filters, but A13 is 3 times more likely expressed in melanocytes than in keratinocytes. Fibroblasts express A13 at intermediate levels. Therefore, A13 is considered as a variable biomarker, not a melanocyte signature biomarker.

Anti-Signature Biomarker Genes

As described herein, anti-signature genes are expressed at markedly lower levels in one cell type compared to other cell types in a group. These genes may code for gene products that interfere with the function of a specific cell type and are suppressed at the normal states. Or, more likely they may not be necessary for a given cell type but are only important for the differentiated status and functions in other cell types. Using the median likelihood method according to this invention, a small number of genes are identified as anti-signature genes, as listed in Table 4. Most of these genes exhibit moderate anti-signature biomarker values. No obvious unifying, functional characteristics are observed in these genes, although they may be useful as diagnostic biomarkers.

Validation of Signature Biomarkers by qRT-PCR

Three signature biomarkers from each of the three human skin cell types in Tables 1-3 were selected for validation by quantitative Real Time reverse transcriptase-polymerase chain reaction (qRT-PCR) amplification. The oligonuclotide primer pairs are shown in Table 7 and the results comparing the DermArray® with qRT-PCR is shown in Table 8. The gene expression profile results are qualitatively concordant between the two methods for all nine of the chosen signature biomarkers. This experiment demonstrates that at least some of the potential robust biomarkers identified using DermArray nylon filters (in the first experiment with keratinocytes, fibroblasts, and melanocytes) have been validated by an independent method and unrelated to nucleic acid hybridization detection methods.

Signature Biomarker Genes for Melanoma Cells

The method of this invention is useful to identify signature and anti-signature biomarker genes for cells in normal as well as abnormal states. DermArray® gene expression experiments are performed in a second experiment using cell culture samples from a primary cutaneous melanoma line (MS7) and a metastatic melanoma line (SKMel-28), besides samples from normal melanocytes. Biomarker genes for these abnormal cells as well as normal melanocytes are identified using the likelihood ratio methods of this invention, as shown in Tables 9-11. Referring to Table 9, the top panel list genes with high Gibbs likelihood values (and hence signature genes of primary cutaneous melanoma). The bottom panel list genes with low Gibbs likelihood values (and hence anti-signature genes of primary cutaneous melanoma). Referring to Table 10, the top panel list genes with high Gibbs likelihood values (and hence signature genes of metastatic melanoma). The bottom panel list genes with low Gibbs Likelihood values (and hence anti-signature genes of metastatic melanoma). Similarly, the top panel of Table 11 list signature (Lip-regulated) genes of normal melanocytes while the bottom panel list the anti-signature genes thereof. For comparison purposes, the simple intensity ratios of these genes are also shown in Tables 9-11. A two-fold change was arbitrarily defined as a significant difference in simple ratio analysis (e.g., >2 or <0.5). There is 72% concordance in genes identified as significantly altered using the Gibbs likelihood method and the simple ratio analysis for the two melanoma cell lines vs normal melanocytes.

Several of the genes identified by the method of this invention are involved in melanin biosynthesis. Decreased expression of two genes in the melanoma cell lines are observed: The likelihood ratio of tyrosinase, the rate-limiting enzyme in melanin biosynthesis, is decreased 2.5 fold in the metastatic melanoma cell line when compared to the normal melanocyte cell line. See, Table 11. Similarly, the expression of tyrosine-related protein 1 (TRP-1) is reduced approximately 2.7 fold in both melanoma cell lines compared to the normal cells. These results are consistent with reduced pigmentation capacity in transformed cell lines. By contrast, and unexpectedly, TRP-2 (tyrosinase-related protein 2 or dopachrome tautomerase), displays increased expression in the melanoma cell lines, especially the primary melanoma line—MS7. See, Tables 9 and 10. TRP-2 has been associated with cell proliferation in addition to its role in melanin production,

Although less well characterized, 6-pyruvoyl-tetrahydropterin synthase (PCD or pterin-4a-carbinolamine dehydratase) also appears to be involved in pigmentation. Masada M. et al., Pigment Cell Res. 3:61-70. Its level of expression is increased three fold in the metastatic melanoma cells when compared to normal melanocytes (Table 10); whereas, the level of its expression in primary melanoma cells remains the same (Table 9). The presence of PCD is necessary for pigment cell formation in Xenopus and dysfunction of this protein is associated with the pigmentation disorder vitiligo. In normal human skin, PCD protein is weakly expressed in the basal layer of the epidermis that consists of keratinocytes and melanocytes. Von Strandmann E P et al. observed that, although only four of 25 benign nevi reacted with PCD-specific antibodies, high protein levels were detectable in melanoma cell lines and 13 of 15 primary malignant melanoma lesions. Von Strandmann et al., Am J. Pathol. 158:2021-2029. Similarly, high levels of PCD expression have been reported in colon tumors and colon cancer cell lines while no expression have been observed in normal colon epithelia. Eskinazi R., et al., Am. J. Pathol., 155:1105-1113.

Many of the biomarker genes identified in melanocytes or melanoma tissue or cell cultures have previously been found in these cell types by other investigators. For example, the CD44 antigen (see Table 11) was observed to have increased expression in the melanocytes relative to the two melanoma cell lines in the DermArray® experiments. Reduced cell surface CD44 levels have been associated with poor prognosis in clinical stage I cutaneous melanoma, and it has been suggested that quantification of CD44 offers a prognostic tool for clinical evaluation. Karjalainen J M et al., Am J. Pathol. 157:957-965. Similarly, CD44 expression in melanomas has been shown to decline in skin lesions with increasing invasive behavior. Harwood, C A et al., Br. J. Dermatol. 135:876-882.

The mechanism by which malignant melanomas are often hypomelanotic or amelanotic is not clear. Although human cutaneous melanoma pathogenesis is believed to be largely due to loss of tumor suppressor function, it is known that some dominant oncogenes alone are capable of reducing pigmentation in murine melanomas. See, Dooley T P, et al., Lab. Animal Sci. 43:48-57; Dooley T P, et al., Oncogene 3:531-535; Wilson R E et al., Cancer Res. 49:711-716. As discussed herein, three biomarker genes identified using the method of this invention are involved in melanin biosynthesis: TRP-1 and Tyrosinase show decreased expression in the 2 melanoma cell lines (Table 11); and, TRP-2 is more highly expressed in primary melanoma cells (Table 9). Reduction in tyrosinase mRNA alone may account for reduced pigmentation in melanomas, as it catalyses the rate-limiting step in melanogenesis. An earlier study by other researchers indicated that TRP-1 expression was decreased in a metastasizing melanoma cell line in comparison to a non-metastasizing cell line. Brem et al., Anticancer Res. 21:1731-1740.

Rab 7 and phosphoinositide 3-kinase (PI3K) are also associated with melanin synthesis. Rab 7 is thought to be a melanosome-associated protein that is involved in the intracellular transport of TRP-1. Gomez P F et al., J. Invest. Dermatol. 117:81-90. As measured by the method of this invention, the expression of Rab 7 appears to be diminished in the melanoma cell lines as shown in Table 11. The regulatory subunit 4 of PI3K demonstrates increased expression in the metastatic melanoma cells (Table 10). Tyrosinase expression is modulated by this kinase. Oka M et al., J. Invest. Dermatol. 115:699-703. Inhibition of the PI3K pathway results in differentiation (and increased melanin production) in B16 melanoma cells. Busca R. et al., J. Biol. Chem. 271:31824-31830. PI3K also appears to be involved in signal transduction required for migration of melanoma cells, regulating formation of actin stress fibers, and alpha V beta 3-integrin-mediated cell adhesion. Metzner B. et al., J. Invest. Dermatol. 107:597-602.

Microphthalmia-associated transcription factor (MITF) has been characterized as a sensitive and specific marker for melanoma. King, R. et al., Am. J. Pathol., 155:731-738. It is a nuclear transcription factor critical for the differentiation and survival of melanocytes and is involved in the transcription of tyrosinase and TRP-1. A decrease in MITF, tyrosinase, and TRP-1 has been observed accompanied by a marked increase in TRP-2 expression, when proliferating cultured neonatal melanocytes are treated with a differentiating agent. Fang D. et al., Pigment Cell Res. 14:132-139. In the DermArray® experiments, MITF is shown to be down regulated in the metastatic melanoma cell line. See, Table 10. The results from Tables 9-11 demonstrate that there is at least partial correlation in the direction of change of expression of MITF, TRP-1 and tyrosinase and, that, a change in expression of TRP-2 is often in the opposite direction.

The Yamaguchi sarcoma viral oncogene homolog (c-yes) expression is elevated in both melanoma cell lines (Table 11). Consistently, earlier immune complex kinase assays and immune blot analysis performed by others using 20 human melanoma and 10 human melanocyte cell lines indicated that the average tyrosine kinase activity of c-yes in most melanoma cell lines is 5-10 fold higher than in melanocyte cell lines. Loganzo F. et al., Oncogene 8:2637-2644.

Further, expression of chorionic gonadotropin (hCG) beta polypeptide is shown to be diminished in the primary melanoma cell line and increased in the metastatic melanoma cell line relative to the normal NHEM cells. See, Table 10. A high frequency of immunoreactive hCG was previously found in patients with melanoma. Ayala A R et al., Am. J. Reprod. Immunol. 3:149-151. In addition, Doi F. has shown that 18 of 24 melanoma cell lines expressed beta-hCG mRNA and that it was expressed in 17/25 melanoma-positive tumor-draining lymph nodes but not detected in normal donor peripheral blood leukocytes and normal lymph nodes. Doi F. et al., Int. J. Cancer 65:454-459. It has been suggested that hCG may be useful molecular marker to define a subset of malignant melanomas.

Therefore, the signature and anti-signature biomarker genes identified using the method of this invention provide validation of many previously identified biomarkers for keratinocytes, melanocytes, and fibroblasts, whether at normal or abnormal states. Further, the method of this invention also identifies certain new biomarker genes that may be useful in pathogenesis studies, molecular diagnostics, and development of therapeutics. Better prognostic value than is currently possible may be achieved with effective biomarkers identified according to this invention.

Using these biomarker genes, diagnostic products may be developed to enhance pathologic characterization of suspected melanocytic lesions and other maladies of skin. Multivariate analyses with multiple biomarkers may be particularly useful in this context. From the genes identified in Tables 9-11, the more than two dozen newly identified potential biomarkers are of particular interest. Each of them has a likelihood ratio of higher than 2.0 or lower than 0.5 and a simple ratio of higher than 2.0 or lower than 0.5. Examples of new signature biomarker genes for the metastatic melanoma cell line include transducer of ERBB2 member 2, Finkel-Biskis-Reilly murine sarcoma virus, RAB6, KIAA0996 protein, homeo box A10, Tax1 binding protein 1, SET binding factor 1, ubiquitination factor E4A, solute carrier family 1 member 3, heterogeneous nuclear ribonucleoprotein A3, and four ESTs (cDNA IDs 471826, 206907, 427657 and 208082), as shown in Table 10. For the primary melanoma cell line, as shown in Table 9, new signature genes include histidyl-tRNA synthetase homolog and an EST (cDNA ID 209841). For normal melanocytes, as shown in Table 11, new signature genes include nidogen 2, erythroid alpha-spectrin 1, afx1 transcription factor, and sarcoma-amplified sequence. New anti-signature genes for metastatic melanomas include fibroblast growth factor 12, intercellular adhesion molecule 2, hematopoietic protein 1, interleukin-1 receptor-associated kinase, and macrophage-associated antigen, as shown in Table 10.

The following examples are offered to illustrate embodiments of the invention are should not be viewed as limiting the scope of the invention.

### EXAMPLE 1

### Microarray Experiments Using Samples from Cultured Cells

In the first experiment, three primary cell types were purchased and cultured: normal human epidermal keratinocytes (NHEK, pooled neonatal; Cascade Biologics), normal human epidermal melanocytes (NHEM, neonatal; Cascade Biologics) and normal human dermal fibroblasts (NHDF, neonatal; BioWhittaker). Cell cultures were plated in 100-mm polystyrene tissue culture dishes (Falcon) with 10 mls media without antibiotic agents, maintained at 37° C., 5% CO₂ and re-fed every two days.

Neonatal NHEK (keratinocyte) cells were initially plated in EpiLife Media (Cascade Biologics) with 60 mM CaCl₂ and were switched at the start of the experiment to 150 mM of CaCl₂ to induce differentiation. Pre-confluent keratinocyte cells were split 1:4, and ten days later (i.e. four days post-confluence) the cells were harvested. Neonatal NHDF (fibroblast) cells were grown in Media 106 (Cascade Biologics), and upon confluence were split 1:5. Six days later (i.e., two days post-confluence) the cells were harvested. Neonatal NHEM (melanocyte) cells were grown in MBM with MGM-3 supplement (BioWhittaker). Pre-confluent melanocytes were split 1:3; and six days later they were harvested. Total RNA samples were extracted using RNeasy Midi Kits (Qiagen).

In the second experiment, a human primary cutaneous melanoma cell line (MS7 from a 66 year old male; obtained from Paola Grammatico, Rome, Italy) and a human metastatic melanoma cell line (SK-Mel 28, from a 51 year old male; obtained from American Type Culture Collection) were cultured. Cells were plated in 100-mm tissue culture treated polystyrene dishes (Falcon) with 10-ml MBM media (Clonetics), MGM-3 growth supplement and Penicillin/streptomycin (Gibco). Cultures were maintained at 37° C., 5% CO₂ and re-fed every other day. The MS7 and SKMel-28 cells were harvested at 100 and 70% confluence, respectively. Total RNA was extracted using RNeasy Midi Kits (Qiagen).

DermArray® DNA microarrays (IntegriDerm ID 1001; www.integriderm.com) were hybridized according to protocols developed by the manufacturer (Invitrogen/ResGen) with certain modifications. Three μg total RNA was utilized as template for a reverse transcriptase reaction (Superscript II, Life Technologies) to create [³²P] dCTP labeled cDNA probes. Reactions were purified by chromatography-columns (Bio-Spin 6, Bio-Rad), and [³²P] incorporation measured by β-counting. New DermArray® filters (not re-used) were pre-washed in boiling 0.5% SDS for 5 min., placed individually in hybridization roller bottles with 5 ml MicroHyb solution, pre-hybridized with 5 μg denatured poly-dA and Cot-1 DNA (Invitrogen/Research Genetics) for 2 hours at 42° C., and then hybridized overnight with individual [³²P] labeled cDNA probes. Arrays were washed for 20 minutes in hybridization bottles at 50° C. with 2×SCC three times; 1% SDS two times; and 0.5×SCC/1% SDS once. Moist filters were wrapped individually with plastic wrap, carefully oriented and exposed to phosphor-storage screens (Packard Instruments) in photographic cassettes for 16 h. Exposed screens were imaged (Cyclone Phosphorimager, Packard Instruments) and tiff files imported into Pathways 2 software (Invitrogen/ResGen) for image alignment and translation of the raw hybridization intensities.

### Example 2

### Normalization of Microarray Data

Hybridization intensities derived from DermArray® filters were normalized before likelihood ratios were calculated to account for, e.g., the differences in total hybridization using different radiolabeled probes.

Particularly, intensities obtained from Pathways 2 software (Invitrogen, see www.invitrogen.com) were standardized as follows:

I_{standardized}=N×(I_{measured}−I_{background})+I_{renormalization}

Here, I_{background} represents background corrections determined using Pathways 2 software. I_{standardized} represents the resulting standardized values. I_{measured} represents the value measured from the array—by Pathways 2 software in this example. Further, I_{renormalization} represents a renormalization factor that is used to shift the resulting values of I_{standardized} back to the proper range of the raw measurements of I_{measured}; I_{renormalization} is designated as ten in this example. Renormalization prevents obtaining negative or zero values of I_{standardized} as a result of normalization. And, N is calculated according to the formula:

N=<I_{control}>/<Iₑₓₚₑᵣᵢₘₑₙₜ>

The background-corrected keratinocyte intensity data was designated as I_{control} in this example; <I_{control}> represents the mean of I_{control}. The background corrected melanocyte or fibroblast intensity data was designated as Iₑₓₚₑᵣᵢₘₑₙₜ; and, <Iₑₓₚₑᵣᵢₘₑₙₜ> represents the mean of Iₑₓₚₑᵣᵢₘₑₙₜ.

### Example 3

### Quantitative RT-PCR

Results from certain microarray expression experiments were verified by quantitative real-time PCR (GeneAmp 5700 Sequence Detector, PE Applied BioSystems). Amplicon formation was quantified by monitoring fluorescence of SYBR® green (PE Applied BioSystems), which can intercalate into double stranded DNA. The following biomarker genes were selected for verification: keratins 5, 14, and 19 for the keratinocyte cells (KRT5, KRT14, KRT19, respectively); vimentin, apolipoprotein D, and collagen 6A for the fibroblast cells (VIM, APOD, COL6A1 respectively); and tyrosinase-related protein 1, silver, and melan A for the melanocyte cells (TRP1, SILV, MLANA respectively). Primer pairs for these genes are listed in Table 7. The same RNA samples were used for the DNA microarray and qRT-PCR analyses. The results of qRT-PCR are shown in Table 8.

Comparing DermArray® intensities and likelihood ratios with qRT-PCR yields (nanograms) in Table 8, one can observe, qualitatively, concordant differential gene expression. Quantitatively, DermArray® signature gene likelihood ratios for certain genes (e.g., SILV) appeared to have estimated the inter-cell type differentials at a lower level than the qRT-PCR experiments. That is, the qRT-PCR experiments tend to yield greater numerical differences. Such results suggest that the likelihood ratio derived from microarray data according to this invention is a stringent selection methodology unlikely to yield false positives.

Examples of genes chosen from the top five percent of the signature biomarker genes according to their likelihood ratios were selected for qRT-PCR verification. Keratinocyte signature biomarkers keratin 5, 14 and 19 were the highest-ranking signature genes in the entire experiment. Silver and melan-A genes were the top signature biomarkers for melanocytes; and, tyrosinase-related protein 1 (TRP1) was another well-known enzyme involved in pigmentation. Vimentin was the highest signature biomarker of fibroblasts. Apolipoprotein D and collagen 6A1 were also selected from the top ten signature genes of fibroblasts cells. As shown in Table 8, all nine of the selected signature biomarker genes identified by DNA microarray analysis were validated by qRT-PCR.

qRT-PCR is an effective method to validate quantitatively the biomarkers discovered using DNA microarrays. However, some of the biomarkers might not validate by qRT-PCR for a variety of reasons. For instance, cross hybridization by a closely-related member of a gene family or superfamily might produce a positive signal in the DNA microarray analysis, but would fail to amplify by the more selective isozyme-specific oligonucleotide primer pair used in the PCR amplification reactions.

Other embodiments are uses of the invention will be apparent to those skilled in the art from consideration of the specification and practice of the invention disclosed herein. All documents cited herein for whatever reason, including U.S. Provisional Application No. 60/354,519, entitled Biomarkers of Human Skin Cells, filed Feb. 4, 2002, are specifically and entirely incorporated by reference. The specification including the examples should be considered exemplary only, are the true scope of the invention defined by the following claims.

TABLE 1
Keratinocyte Signature Biomarker

| Function | cDNA ID | Gene | Symbol | K | F | M | Reference |
|---|---|---|---|---|---|---|---|
| Keratins | 183602 | keratin 14 | KRT14 | 403.76 | 0.01 | 0.00 | Purkis PE, 1990 |
| | 592540 | keratin 5 | KRT5 | 172.26 | 0.02 | 0.01 | Purkis PE, 1990 |
| | 810131 | keratin 19 | KRT19 | 93.10 | 0.03 | 0.02 | Stasiak PC, 1989 |
| | 855521 | keratin 18 | KRT18 | 24.70 | 0.09 | 0.07 | Abo S, 1999 |
| | 843321 | keratin 7 | KRT7 | 13.90 | 0.21 | 0.11 | Williams GR, 1997 |
| | 897781 | keratin 8 | KRT8 | 11.90 | 0.20 | 0.13 | Cheng C, 1993 |
| | 592111 | keratin 6B | KRT6B | 8.57 | 0.24 | 0.22 | Tennenbaum T, 1996 |
| | 342008 | keratin 13 | KRT13 | 8.32 | 0.20 | 0.23 | van Rossum MM, 2000 |
| Cornification | 813614 | small proline-rich protein 1B (cornifin) | SPRR1B | 80.28 | 0.09 | 0.10 | Marvin KW, 1992 |
| | 729942 | small proline-rich protein 2C | SPRR2C | 2.04 | 0.63 | 0.69 | — |
| Annexins | 810813 | S100 A2 | S100A2 | 37.01 | 0.06 | 0.05 | Deshpande R, 2000 |
| | 1459376 | S100 A9 (calgranulin B) | S100A9 | 4.07 | 0.35 | 0.44 | Clark BR, 1990 |
| | 810612 | S100 A11 (calgizzarin) | S100A11 | 2.75 | 0.50 | 0.56 | Robinson NA, 1997 |
| | 756595 | S100 A10 (calpactin I light chain) | S100A10 | 2.71 | 0.56 | 0.54 | Robinson NA, 1997 |
| | 562729 | S100 A8 (calgranulin A) | S100A8 | 1.79 | 0.77 | 0.73 | Clark BR, 1990 |
| ECM & adhesion | 359747 | (galectin 7) | LGALS7 | 25.44 | 0.08 | 0.07 | Madsen P, 1995 |
| | 240961 | desmoplakin | DSP | 12.29 | 0.16 | 0.14 | Haftek M, 1991 |
| | 1472775 | collagen, type VIII, alpha 1 | COL8A1 | 6.93 | 0.28 | 0.25 | — |
| | 897720 | trophinin | TRO | 3.83 | 0.41 | 0.42 | — |
| | 1609966 | chondroitin sulfate proteoglycan 3 | CSPG3 | 2.75 | 0.49 | 0.58 | — |
| | 229692 | collagen, type IV, alpha 4 | COL4A4 | 2.40 | 0.61 | 0.57 | — |
| Cytokines | 182661 | activin A receptor type II-like 1 | ACVRL1 | 5.06 | 0.31 | 0.35 | — |
| | 49665 | endothelin receptor type B | EDNRB | 2.59 | 0.67 | 0.75 | Yohn JJ, 1994 |
| | 84295 | interleukin 1 receptor antagonist | IL1RN | 1.78 | 0.71 | 0.73 | Hammerberg C, 1998 |
| Development | 230882 | paired box gene 6 (aniridia, keratitis) | PAX6 | 5.35 | 0.33 | 0.30 | — |
| | 813611 | homeobox D4 | HOXD4 | 4.29 | 0.54 | 0.61 | — |
| | 150702 | homeobox B5 | HOXB5 | 3.00 | 0.49 | 0.51 | — |
| | 594540 | patched (Drosophila) homolog | PTCH | 2.96 | 0.54 | 0.54 | Wikonkal NM, 1999 |
| Transcription | 590148 | (zinc finger protein 131) | ZNF131 | 2.06 | 0.65 | 0.65 | — |
| | 341317 | (zinc finger protein - EST) | KIAA0222 | 1.80 | 0.70 | 0.73 | — |
| | 309864 | jun B proto-oncogene | JUNB | 1.73 | 0.73 | 0.74 | Mauviel A, 1996 |
| | 364510 | special AT-rich sequence binding 1 | SATB1 | 1.89 | 0.70 | 0.68 | — |
| Proteases | 378813 | antileukoproteinase | SLPI | 10.35 | 0.17 | 0.19 | Gibbs S, W 2000 |
| | 1474174 | matrix metalloproteinase 2 | MMP2 | 4.74 | 0.46 | 0.27 | Kobayashi T, 1998 |
| | 340898 | ubiquitin specific protease 16 | USP16 | 3.02 | 0.48 | 0.52 | — |
| Enzymes | 588500 | pyrroline-5-carboxylate synthetase | PYCS | 13.29 | 0.16 | 0.12 | — |
| | 1631713 | neural exp. devel. down-reg. 5 | NEDD5 | 6.90 | 0.25 | 0.26 | — |
| | 25679 | steroid-5-alpha-reductase, 1 | SRD5A1 | 5.13 | 0.40 | 0.38 | Milewich L, 1988 |
| | 810854 | ribonuclease P (30kD) | RPP30 | 4.54 | 0.49 | 0.25 | — |
| | 897788 | protein tyrosine phosphatase, rec F | PTPRF | 3.67 | 0.54 | 0.33 | — |
| | 67625 | lipase, endothelial | LIPG | 3.67 | 0.47 | 0.39 | — |
| | 591907 | ras homolog gene | ARHD | 3.19 | 0.53 | 0.43 | — |
| | 810445 | valyl-tRNA synthetase 2 | VARS2 | 2.52 | 0.51 | 0.63 | — |
| | 183440 | arylsulfatase A | ARSA | 2.42 | 0.61 | 0.62 | — |
| | 359661 | monoamine oxidase A | MAOA | 2.38 | 0.61 | 0.57 | Schallreuter KU, 1996 |
| | 196992 | aldo-keto reductase 1C1 | AKR1C1 | 2.10 | 0.74 | 0.56 | — |
| | 502198 | protein phosphatase 1, regulatory 3C | PPP1R3C | 1.97 | 0.74 | 0.61 | — |
| | 842980 | devel regulated GTP-binding 1 | DRG1 | 1.90 | 0.73 | 0.70 | — |
| | 66564 | 3-hydroxybutyrate dehydrogenase | BDH | 1.85 | 0.66 | 0.75 | — |
| Other | 183476 | adipose most abundant transcript | APM1 | 12.88 | 0.17 | 0.16 | — |
| | 210873 | pancreatic polypeptide 2 | PPY2 | 10.78 | 0.18 | 0.16 | — |
| | 586990 | solute carrier 11A2 | SLC11A2 | 6.21 | 0.27 | 0.28 | — |
| | | (transforming sequence, thyroid-1- | | | | | |
| | 461287 | EST) | D10S170 | 3.27 | 0.49 | 0.44 | — |
| | 1580342 | cardiac ankyrin repeat protein | CARP | 2.93 | 0.53 | 0.49 | — |
| | 1486082 | heparin-binding GF binding protein | HBP17 | 2.42 | 0.56 | 0.61 | — |
| | 135449 | Ewing sarcoma breakpointregion 1 | EWSR1 | 1.93 | 0.62 | 0.75 | — |
| | 415613 | (DHHC1 protein - EST) | LOC51304 | 1.82 | 0.68 | 0.74 | — |
| EST | 415281 | EST | — | 7.41 | 0.23 | 0.25 | — |
| | 460258 | EST | — | 3.13 | 0.46 | 0.51 | — |
| | 1501931 | EST - solute carrier family 22A11 | — | 3.05 | 0.47 | 0.52 | — |
| | 415235 | EST | — | 2.36 | 0.46 | 0.74 | — |
| | 67330 | EST | — | 2.22 | 0.66 | 0.59 | — |
| | 460247 | EST | — | 1.95 | 0.61 | 0.75 | — |
| | 1522679 | EST | — | 1.89 | 0.67 | 0.71 | — |
| | 210803 | EST | — | 1.74 | 0.75 | 0.71 | — |
| | 378420 | EST | — | 1.73 | 0.72 | 0.74 | — |

TABLE 2
Fibroblast Signature Biomarkers

| Function | cDNA ID | Gene | Symbol | K | F | M | Reference |
|---|---|---|---|---|---|---|---|
| ECM & adhesion | 840511 | vimentin | VIM | 0.04 | 6.98 | 0.52 | Nishio K, 2001 |
| | 839991 | collagen, type I, alpha 2 | COL1A2 | 0.32 | 5.35 | 0.33 | Ghosh AK, 2000 |
| | 263716 | collagen, type VI, alpha 1 | COL6A1 | 0.51 | 3.50 | 0.62 | Lamande SR,1998 |
| | 50483 | fibulin 5 (EGF-related) | FBLN5 | 0.63 | 2.95 | 0.39 | — |
| | 774409 | endoglin | ENG | 0.60 | 1.97 | 0.76 | Matsubara S, 2000 |
| cytokines | 753620 | insulin-like growth factor binding 6 | IGFBP6 | 0.40 | 4.11 | 0.39 | Martin JL, 1994 |
| | 52096 | platelet-derived GF receptor, alpha | PDGFRA | 0.56 | 2.39 | 0.62 | Czuwara-Ladykowska J, 2001 |
| | 767202 | latent TGF beta binding protein 2 | LTBP2 | 0.75 | 2.22 | 0.51 | Bashir MM, 1996 |
| | 244355 | interleukin 2 receptor, gamma | IL2RG | 0.71 | 1.75 | 0.74 | — |
| translation | 34849 | eukaryotic translation elongation factor 2 | EEF2 | 0.41 | 3.71 | 0.44 | — |
| | 788334 | mitochondrial ribosomal protein L23 | MRPL23 | 0.53 | 2.30 | 0.69 | — |
| | 51981 | ribosomal protein L7a | RPL7A | 0.69 | 1.91 | 0.69 | — |
| transport | 159608 | apolipoprotein D | APOD | 0.40 | 3.68 | 0.46 | Provost PR, 1991 |
| | 743804 | SEC23-like protein B | SEC23B | 0.27 | 3.13 | 0.74 | — |
| | 502151 | solute carrier family 16A3 | SLC16A3 | 0.56 | 2.09 | 0.74 | — |
| enzymes | 245990 | metallothionein 1F | MT1F | 0.43 | 5.64 | 0.19 | — |
| | 700527 | glutaredoxin (thioltransferase) | GLRX | 0.26 | 4.73 | 0.44 | Okuda M, 2001 |
| | 214162 | metallothionein 1H | MT1H | 0.50 | 4.31 | 0.28 | — |
| | 813654 | tyrosine hydroxylase | TH | 0.62 | 1.95 | 0.73 | Ramchand CN, 1995 |
| | 882522 | argininosuccinate synthetase | ASS | 0.69 | 1.90 | 0.68 | Saheki T, 1982 |
| other | 1455976 | interferon induced transmembrane 2 | IFITM2 | 0.34 | 3.47 | 0.57 | — |
| | 1473274 | myosin regulatory light chain 2 | MYRL2 | 0.54 | 3.41 | 0.37 | Kumar CC, 1992 |
| | 868368 | thymosin, beta 4X | TMSB4X | 0.63 | 3.37 | 0.30 | Zalvide JB, 1995 |
| | 760299 | Dickkopf homolog 3 | DKK3 | 0.62 | 3.31 | 0.33 | — |
| | 840687 | episialin (mucin-related) | — | 0.55 | 2.13 | 0.73 | — |
| | 261204 | high mobility group protein I-C | HMG1C | 0.64 | 2.05 | 0.67 | — |
| | 726086 | tissue factor pathway inhibitor 2 | TFPI2 | 0.59 | 2.02 | 0.73 | Izumi H, 2000 |
| | 244307 | plasminogen activator inhibitor, type I | PAI1 | 0.72 | 2.00 | 0.66 | Mu XC, 1998 |
| | 131268 | growth factor receptor-bound protein 14 | GRB14 | 0.76 | 1.96 | 0.60 | — |
| | 304908 | E2F transcription factor 3 | E2F3 | 0.72 | 1.93 | 0.65 | Flores AM, 1998 |
| EST | 1049033 | EST | — | 0.49 | 2.47 | 0.67 | — |
| | 378458 | EST | — | 0.66 | 1.93 | 0.71 | — |

TABLE 3
Melanocyte Signature Biomarkers

| Function | cDNA ID | Gene | Symbol | K | F | M | Reference |
|---|---|---|---|---|---|---|---|
| pigmentation | 266361 | melan-A | MLANA | 0.06 | 0.05 | 38.79 | Busam KJ, 1999 |
| | 291448 | silver | SILV | 0.06 | 0.04 | 39.48 | Solano F, 2000 |
| | 271985 | tyrosinase | TYR | 0.69 | 0.69 | 4.72 | Jimenez-Cervantes 2001 |
| | 773330 | transmembrane glycoprotein | GPNMB | 0.46 | 0.35 | 3.97 | Weterman MA, 1995 |
| | 1933036 | ocular albinism 1 | OA1 | 0.43 | 0.44 | 3.85 | Shen B, 2001 |
| | 269791 | tyrosinase-related protein 2 | DCT | 0.48 | 0.49 | 3.19 | Jimenez-Cervantes C, 2001 |
| ECM & adhesion | 855910 | galectin 3 | LGALS3 | 0.30 | 0.25 | 6.36 | — |
| | 813533 | syndecan binding protein (syntenin) | SDCBP | 0.39 | 0.60 | 3.07 | — |
| | 755975 | dystroglycan 1 | DAG1 | 0.71 | 0.75 | 1.74 | — |
| cytokines | 788832 | prostate differentiation factor | PLAB | 0.59 | 0.69 | 2.12 | — |
| | 797048 | bone morphogenetic protein 4 | BMP4 | 0.67 | 0.74 | 1.84 | Jin EJ, 2001 |
| | 215000 | vasoactive intestinal peptide receptor 1 | VIPR1 | 0.76 | 0.76 | 1.64 | Bellan C, 1992 |
| enzymes | 711918 | glutaminyl cyclotransferase | QPCT | 0.17 | 0.19 | 10.19 | — |
| | 1471841 | ATPase, Na+/K+ transporting, alpha 1 | ATP1A1 | 0.56 | 0.70 | 2.20 | — |
| | 854760 | protein kinase, cAMP-dependent, I, alpha | PRKAR1A | 0.59 | 0.74 | 2.02 | — |
| | 73638 | protein tyrosine phosphatase IVA, 2 | PTP4A2 | 0.63 | 0.73 | 1.94 | — |
| | 278501 | fyn oncogene (tyrosine kinase) | FYN | 0.73 | 0.67 | 1.86 | — |
| | 809421 | 6-pyruvoyl-tetrahydropterin synthase | PCBD | 0.70 | 0.73 | 1.79 | — |
| | 297061 | dihydropyrimidinase | DPYS | 0.74 | 0.74 | 1.70 | — |
| other | 234237 | pirin | PIR | 0.39 | 0.33 | 4.61 | — |
| | 726846 | het. nuclear ribonucleoprotein L | HNRPL | 0.45 | 0.30 | 4.31 | Castelli C, 1999 |
| | 810142 | major histocompatibility complex, 1-C | HLA-C | 0.38 | 0.43 | 3.93 | — |
| | 856454 | 4F2 antigen heavy chain/solute carrier 3 | SLC3A2 | 0.71 | 0.21 | 3.60 | — |
| | 789182 | proliferating cell nuclear antigen | PCNA | 0.62 | 0.42 | 2.90 | Iyengar B, 1994 |
| | 265102 | abl-interactor 2b | ABI2B | 0.68 | 0.61 | 2.48 | — |
| | 265680 | coxsackie virus and adenovirus receptor | CXADR | 0.56 | 0.76 | 2.04 | — |
| | 843134 | prostatic binding protein | PBP | 0.58 | 0.74 | 2.03 | — |
| | 22731 | proteolipid protein 1 | PLP | 0.73 | 0.62 | 1.99 | — |
| | 897642 | v-abl oncogene 1 | ABL1 | 0.65 | 0.72 | 1.93 | — |
| | 415870 | ets2 repressor factor | ERF | 0.71 | 0.70 | 1.82 | — |
| | 268188 | proline-rich Gla 1 | PRRG1 | 0.72 | 0.74 | 1.77 | — |
| | 1475662 | axin1 up-regulated | AXUD1 | 0.75 | 0.75 | 1.67 | — |
| | 48631 | Voltage-gated K channel, beta subunit | — | 0.74 | 0.68 | 1.84 | — |
| EST | 712604 | EST | — | 0.38 | 0.42 | 4.03 | — |
| | 267859 | EST | — | 0.64 | 0.56 | 2.34 | — |
| | 320588 | EST | — | 0.68 | 0.60 | 2.14 | — |
| | 1048698 | EST - vaccinia-related kinase 3 | — | 0.60 | 0.70 | 2.07 | — |
| | 305843 | EST | — | 0.69 | 0.71 | 1.85 | — |

TABLE 4
Keratinocyte Anti-signature Biomarkers

| Function | CDNA ID | Gene | Symbol | K | F | M | Reference |
|---|---|---|---|---|---|---|---|
| adhesion | 1435862 | Antigen (antibodies 12E7, F21 and O13) | MIC2 | 0.25 | 1.49 | 1.72 | — |
| structural | 629896 | Microtubule-associated protein 1B | MAP1B | 0.28 | 1.74 | 1.41 | — |
| transcription | 949928 | Monocytic leukemia zinc finger protein | MOZ | 0.42 | 1.61 | 1.22 | — |
| structural | 384851 | Clathrin heavy chain 1 | CLTCL1 | 0.48 | 1.50 | 1.21 | — |
| enzyme | 897822 | Similar to spleen tyrosine kinase | SYK | 0.51 | 1.32 | 1.37 | Dong G, 2001 |
| cell cycle | 841641 | G1/S-Specific cyclin D1 | CCND1 | 0.54 | 1.35 | 1.25 | Dong G, 2001 |
| — | 624744 | IGF-II mRNA-binding protein 3 | KOC1 | 0.56 | 1.33 | 1.23 | Runge S, 2000 |
| EST | 53371 | EST | — | 0.56 | 1.36 | 1.21 | — |
| — | 203003 | Non-metastatic cells 4 | NME4 | 0.57 | 1.32 | 1.22 | — |
| enzyme | 359038 | TC10-like Rho GTPase | TCL | 0.57 | 1.31 | 1.24 | — |
| EST | 1467936 | EST | — | 0.60 | 1.29 | 1.21 | — |
| transcription | 898195 | Myelin gene expression factor 2 | MEF2 | 0.66 | 1.20 | 1.20 | — |
| transcription | 345621 | CAAX box 1 | CXX1 | 0.66 | 1.19 | 1.21 | — |

TABLE 5
Fibroblast Anti-signature Biomarkers

| Function | cDNA ID | Gene | Symbol | K | F | M | Reference |
|---|---|---|---|---|---|---|---|
| Enzyme | 136235 | Glutathione S-transferase pi | GSTP1 | 1.38 | 0.40 | 1.49 | Hour TC, 1999 |
| Enzyme | 823590 | Sialyltransferase | STHM | 1.34 | 0.41 | 1.49 | Berger EG, 1985 |
| Enzyme | 82734 | Fatty acid-coenzyme A ligase, long chain 2 | FACL2 | 1.27 | 0.47 | 1.46 | — |
| Calcium | 27516 | Calcium modulating ligand | CAML | 1.50 | 0.49 | 1.20 | — |
| EST | 197520 | Nuclear receptor coactivator 3 (AIBI) | — | 1.30 | 0.60 | 1.20 | — |

TABLE 6
Melanocyte Anti-signature Biomarkers

| Function | cDNA ID | Gene | Symbol | K | F | M | Reference |
|---|---|---|---|---|---|---|---|
| Adhesion | 23185 | Hexabrachion | HXB | 1.26 | 2.04 | 0.37 | Le Poole IC, 1997 |
| Protease | 714106 | Plasminogen activator, urokinase | PLAU | 1.32 | 1.31 | 0.53 | de Vries TJ, 1996 |
| Transcription | 840944 | Early growth response 1 | EGR1 | 1.21 | 1.33 | 0.58 | Jean S, 2001 |
| Translation | 878681 | Ribosomal protein L30 | RPL30 | 1.21 | 1.29 | 0.60 | — |

Table 7

Primers for qRT-PCR

| | | |
|---|---|---|
| Gene | Forward | Reverse |

| | | | | |
|---|---|---|---|---|
| KRT5 | TGAGATGAACCGGATGATCCA | (SEQ ID NO 1) | GCAGATTGGCGCACTGTTT | (SEQ ID NO 10) |
| KRT14 | AGCAGCAGAACCAGGAGTACAAG | (SEQ ID NO 2) | GGCGGTAGGTGGCGATCT | (SEQ ID NO 11) |
| KRT19 | CAGGTCAGTGTGGAGGTGGAT | (SEQ ID NO 3) | TCGCATGTCACTCAGGATCTTG | (SEQ ID NO 12) |
| APOD | CTGGCCACCGACTATGAGAAC | (SEQ ID NO 4) | AAAATCCACGTGAAAAAGTTGGA | (SEQ ID NO 13) |
| COL6A1 | TGACCCCGACCTCAGAGAGT | (SEQ ID NO 5) | CCGTTAATCTCGAGGGTCTTGA | (SEQ ID NO 14) |
| VIM | ACACCCTGCAATCTTTCAGACA | (SEQ ID NO 6) | GATTCCACTTTGCGTTCAAGGT | (SEQ ID NO 15) |
| MLANA | ACTTCATCTATGGTTACCCCAAGAA | (SEQ ID NO 7) | GATCCCAGCGGCCTCTTC | (SEQ ID NO 16) |
| SILV | TGGGACAGGCAGGGCA | (SEQ ID NO 8) | TCCCCGGCGATGGTAGA | (SEQ ID NO 17) |
| TRP1 | GCCCCACAGCCCTCAGTA | (SEQ ID NO 9) | AAGCGCAAGGGCCAGAC | (SEQ ID NO 18) |

TABLE 8
Verification By qRT-PCR

| | DermArray ® DNA Array | | | | | | qRT-PCR | | |
|---|---|---|---|---|---|---|---|---|---|
| Gene | I_{K} | I_{F} | I_{M} | L_{K} | L_{F} | L_{M} | K [ng] | F [ng] | M [ng] |
| KRT5 | 3301.3 | 23.8 | 17.7 | 172.3 | 0.0 | 0.0 | 137.0 | 0.0 | 0.0 |
| KRT14 | 7106.5 | 19.5 | 16.2 | 403.8 | 0.0 | 0.0 | 90.0 | 0.0 | 2.7 |
| KRT19 | 2424.6 | 32.4 | 19.8 | 93.1 | 0.0 | 0.0 | 3.9 | 0.2 | 0.7 |
| VIM | 44.0 | 1976.8 | 522.7 | 0.0 | 7.0 | 0.5 | 17.8 | 252.2 | 104.5 |
| APOD | 23.1 | 89.9 | 25.8 | 0.4 | 3.7 | 0.5 | 0.0 | 29.8 | 5.2 |
| COL6A1 | 20.3 | 86.4 | 23.4 | 0.5 | 3.5 | 0.6 | 0.0 | 17.0 | 1.0 |
| TRP1 | 15.7 | 22.1 | 37.1 | 0.6 | 0.8 | 2.0 | 0.1 | 0.0 | 154.7 |
| SILV | 30.8 | 19.4 | 990.8 | 0.1 | 0.0 | 39.5 | 0.0 | 0.0 | 106.2 |
| MLANA | 21.4 | 16.6 | 730.9 | 0.1 | 0.0 | 38.8 | 0.0 | 0.0 | 59.7 |

TABLE 9
Primary Cutaneous Melanoma Biomarkers (MS7)

| | | Gibbs Likelihood | | | Simple Ratio | | |
|---|---|---|---|---|---|---|---|
| cDNA ID | Gene | N | P | M | P/N | P/M | Reference |
| 770957 | Dihydropyrimidine dehydrogenase | 0.67 | 2.85 | 0.38 | 2.30 | 1.58 | |
| 80924 | Histidyl-tRNA synthetase homolog | 0.54 | 2.82 | 0.50 | 4.04 | 2.57 | |
| 209841 | EST | 0.52 | 2.29 | 0.70 | 2.54 | 2.07 | |
| 269791 | TRP-2 | 0.25 | 2.18 | 1.19 | 5.24 | 1.45 | Aroca P. 1990 [25] |
| 767049 | Proteasome 26S subunit, ATPase, 6 | 0.75 | 2.16 | 0.53 | 1.82 | 2.25 | |
| 450777 | MYC-associated zinc finger protein | 0.48 | 2.10 | 0.84 | 1.63 | 0.60 | |
| 840511 | Vimentin | 1.85 | 0.39 | 1.10 | 0.38 | 0.40 | Wang R, 2001 [26] |

TABLE 10
Metastatic Melanoma Biomarkers (SKMel-28)

| | | Gibb's Likelihood | | | Simple Ratio | | |
|---|---|---|---|---|---|---|---|
| CDNA ID | Gene | N | P | M | M/N | M/P | Reference |
| 563574 | FSHD region gene 1 | 0.35 | 0.48 | 3.83 | 0.96 | 2.52 | |
| 840775 | Transducer of ERBB2, 2 | 0.45 | 0.45 | 3.45 | 4.25 | 3.53 | |
| 471826 | EST | 0.61 | 0.33 | 3.35 | 2.36 | 3.69 | |
| 141747 | Finkel-Biskis-Reilly murine sarcoma virus | 0.46 | 0.49 | 3.19 | 3.14 | 2.87 | |
| 45544 | Transgelin 2 | 0.51 | 0.50 | 2.95 | 1.94 | 1.84 | |
| 172440 | RAB6 | 0.52 | 0.50 | 2.93 | 2.42 | 2.30 | |
| 298104 | KIAA0996 protein | 0.60 | 0.42 | 2.93 | 2.47 | 3.44 | |
| 1592006 | Homeo box A10 | 0.44 | 0.58 | 2.92 | 2.56 | 2.05 | |
| 898109 | Tax1 binding protein 1 | 0.61 | 0.42 | 2.91 | 2.16 | 2.96 | |
| 126674 | SET binding factor 1 | 0.57 | 0.46 | 2.91 | 3.31 | 3.22 | |
| 206907 | EST (Maternally expressed 3) | 0.48 | 0.56 | 2.87 | 3.42 | 3.20 | |
| 1160723 | LIM domain kinase 2 | 0.73 | 0.37 | 2.70 | 0.93 | 1.25 | |
| 782587 | Ubiquitination factor E4A | 0.55 | 0.54 | 2.66 | 2.61 | 2.64 | |
| 448088 | EST | 0.45 | 0.66 | 2.65 | 2.37 | 1.75 | |
| 427657 | EST | 0.72 | 0.39 | 2.64 | 2.17 | 2.95 | |
| 813678 | Solute carrier family 1, member 3 | 0.59 | 0.51 | 2.62 | 2.66 | 3.08 | |
| 810567 | Guanine nucleotide regulatory factor | 0.59 | 0.53 | 2.58 | 1.02 | 1.15 | |
| 757144 | Heterogeneous nuclear protein | 0.50 | 0.63 | 2.54 | 2.40 | 2.07 | |
| 208082 | EST | 0.50 | 0.64 | 2.53 | 2.73 | 2.99 | |
| 788511 | Ribosomal protein S6 kinase, 90kD, polypeptide 1 | 0.59 | 0.55 | 2.52 | 1.29 | 0.73 | |
| 259973 | Chorionic gonadotropin, beta polypeptide | 0.70 | 0.45 | 2.52 | 1.27 | 1.79 | Doi F, 1996 [16] |
| 207087 | SCN Circadian Oscillatory Protein | 0.39 | 0.78 | 2.51 | 2.69 | 1.94 | |
| 898218 | Insulin-like growth factor binding protein 3 | 0.73 | 0.44 | 2.49 | 1.67 | 2.32 | |
| 856174 | Phosphoinositide-3-kinase, regulatory subunit 4, p150 | 0.54 | 0.61 | 2.47 | 3.21 | 3.08 | Oka M, 2000 [8] |
| 782545 | Hemogen - EST | 0.69 | 0.47 | 2.46 | 2.22 | 2.84 | |
| 1468466 | EST | 0.58 | 0.58 | 2.46 | 2.47 | 2.21 | |
| 447088 | EST | 0.64 | 0.52 | 2.46 | 1.55 | 2.02 | |
| 159608 | Apolipoprotein D | 0.58 | 0.58 | 2.44 | 2.25 | 2.78 | |
| 795191 | X-prolyl aminopeptidase-like | 0.61 | 0.56 | 2.43 | 1.77 | 1.79 | |
| 1590269 | Solute carrier family 2, member 4 | 0.57 | 0.61 | 2.40 | 2.73 | 2.31 | |
| 1571632 | EST | 0.71 | 0.48 | 2.38 | 2.05 | 2.83 | |
| 773392 | Cartilage linking protein 1 | 0.42 | 0.79 | 2.37 | 3.31 | 2.25 | |
| 45556 | MAP/microtubule affinity-regulating kinase 3 | 0.61 | 0.58 | 2.36 | 1.83 | 1.91 | |
| 1572233 | Putative G0/G1 switch gene 2 | 0.66 | 0.54 | 2.35 | 1.93 | 2.40 | |
| 714210 | Putative nucleic acid binding protein RY-1 | 0.52 | 0.69 | 2.33 | 2.03 | 1.57 | |
| 591143 | EST | 0.71 | 0.53 | 2.25 | 2.30 | 2.28 | |
| 245990 | RNA helicase-related protein | 0.50 | 0.76 | 2.24 | 2.36 | 2.03 | |
| 171936 | Hippocalcin | 0.62 | 0.62 | 2.22 | 2.60 | 2.31 | |
| 46518 | Dystrobrevin, alpha | 0.59 | 0.65 | 2.22 | 2.03 | 2.14 | |
| 378458 | EST | 0.80 | 0.47 | 2.22 | 1.42 | 2.47 | |
| 813654 | Tyrosine hydroxylase | 0.78 | 0.48 | 2.22 | 1.37 | 1.58 | |
| 42627 | Coagulation factor C homolog | 0.63 | 0.62 | 2.20 | 2.67 | 2.13 | |
| 745387 | Tumor protein p53-binding protein, 1 | 0.51 | 0.77 | 2.15 | 2.26 | 1.94 | |
| 753321 | EST | 0.75 | 0.53 | 2.14 | 1.31 | 1.55 | |
| 877827 | Ribosomal protein S27a | 0.75 | 0.54 | 2.12 | 2.97 | 3.73 | Santa Cruz DJ, 1997 [27] |
| 24838 | EST | 0.52 | 0.77 | 2.12 | 2.11 | 1.72 | |
| 703707 | Aspartate beta-hydroxylase | 0.80 | 0.51 | 2.09 | 1.41 | 2.06 | |
| 510032 | Putative receptor protein | 0.81 | 0.51 | 2.09 | 2.74 | 3.92 | |
| 471729 | Ornithine transporter, mitochondrial | 0.85 | 0.47 | 2.08 | 2.26 | 3.57 | |
| 823691 | Cyclin G2 | 0.87 | 0.49 | 2.01 | 2.41 | 3.69 | |
| 251618 | Dynein, cytoplasmic, heavy polypeptide 1 | 0.89 | 0.48 | 1.99 | 1.42 | 1.96 | |
| 324437 | GRO1 oncogene | 0.84 | 0.57 | 1.96 | 1.07 | 1.80 | Bordoni R, 1990 [28] |
| 813402 | Renin | 0.46 | 1.00 | 1.85 | 2.36 | 1.46 | |
| 215000 | Vasoactive intestinal peptide receptor 1 | 1.04 | 1.77 | 0.46 | 1.22 | 0.82 | Bellan C, 1992 [29] |
| 379771 | Keratin 1 (epidermolytic hyperkeratosis) | 1.54 | 1.24 | 0.46 | 0.47 | 0.36 | |
| 151501 | TEK tyrosine kinase | 1.69 | 1.12 | 0.44 | 0.32 | 0.54 | |
| 50930 | Fibroblast growth factor 12 | 1.40 | 1.39 | 0.44 | 0.37 | 0.46 | |
| 130201 | Intercellular adhesion molecule 2 | 1.67 | 1.23 | 0.43 | 0.37 | 0.48 | |
| 854284 | Hematopoietic protein 1 | 1.28 | 1.52 | 0.43 | 0.39 | 0.38 | |
| 277229 | Toll-like receptors | 1.75 | 1.11 | 0.43 | 0.46 | 0.70 | |
| 214985 | Nuclear domain 10 protein | 1.41 | 1.44 | 0.40 | 0.41 | 0.39 | |
| 379200 | Interleukin-1 receptor-associated kinase 1 | 1.27 | 1.62 | 0.39 | 0.36 | 0.37 | |
| 278570 | Microphthalmia-associated transcription factor | 1.20 | 1.72 | 0.39 | 0.40 | 0.34 | King R, 1999 [11] |
| 727292 | Macrophage-associated antigen | 1.94 | 1.21 | 0.30 | 0.20 | 0.31 | |

TABLE 11
Normal Melanocyte Biomarkers

| | | Gibb's Likelihood | | | Simple Ratio | | |
|---|---|---|---|---|---|---|---|
| cDNA ID | Gene | N | P | M | N/P | N/M | Reference |
| 754479 | Hypothetical protein, expressed in osteoblast | 3.86 | 0.36 | 0.46 | 1.68 | 1.59 | |
| 897626 | RAB7 | 3.09 | 0.44 | 0.54 | 3.28 | 2.87 | Gomez P. 2001 [7] |
| 768344 | TRP-1 | 2.70 | 0.52 | 0.60 | 2.71 | 2.64 | del Marmol V, 1993 [20] |
| 214572 | Cyclin-dependent kinase 6 | 2.64 | 0.57 | 0.53 | 2.46 | 2.57 | Tang L, 1999 [21] |
| 754093 | Nidogen-2 | 2.58 | 0.55 | 0.57 | 3.36 | 3.43 | |
| 427750 | Erythroid alpha-spectrin 1 | 2.54 | 0.49 | 0.65 | 2.82 | 2.30 | |
| 70349 | Afx1 transcription factor | 2.53 | 0.63 | 0.51 | 2.06 | 2.04 | |
| 35105 | Sarcoma amplified sequence | 2.52 | 0.60 | 0.54 | 2.30 | 2.54 | |
| 210575 | Visinin-like 1 | 2.47 | 0.46 | 0.70 | 2.68 | 2.08 | |
| 221846 | Checkpoint suppressor 1 | 2.45 | 0.63 | 0.53 | 2.23 | 2.51 | |
| 882588 | Putative nuclear protein | 2.37 | 0.52 | 0.67 | 2.45 | 2.07 | |
| 856454 | Solute carrier family 3 member 2 | 2.33 | 0.63 | 0.57 | 2.52 | 2.22 | Dixon WT, 1990 [22] |
| 713145 | CD44 antigen | 2.32 | 0.53 | 0.67 | 2.69 | 2.04 | Karjalainen JM, 2000 [1] |
| 756968 | Ephrin-B1 | 2.28 | 0.66 | 0.56 | 2.14 | 2.45 | |
| 144786 | Biglycan | 2.24 | 0.59 | 0.65 | 2.01 | 1.76 | |
| 73638 | Protein tyrosine phosphatase type IVA, member 2 | 2.23 | 0.61 | 0.63 | 2.25 | 2.27 | |
| 754378 | Prostaglandin E synthase | 2.20 | 0.53 | 0.73 | 2.52 | 1.99 | |
| 23173 | Mitogen-activated protein kinase 10 | 2.20 | 0.55 | 0.70 | 2.29 | 2.01 | |
| 856535 | Methylenetetrahydrofolate dehydrogenase | 2.16 | 0.80 | 0.48 | 1.99 | 3.92 | |
| 815737 | ATP synthase, H+ transporting, mitochondrial F1 alpha 1 | 2.12 | 0.76 | 0.53 | 1.92 | 2.46 | |
| 47142 | Peroxisomal biogenesis factor 12 | 2.08 | 0.94 | 0.41 | 2.08 | 3.77 | |
| 767638 | Pleiomorphic adenoma gene 1 | 2.07 | 0.82 | 0.50 | 1.28 | 2.11 | |
| 592359 | HLA class II region expressed gene KE4 | 2.02 | 0.42 | 0.97 | 1.04 | 0.99 | |
| 267864 | RANTES | 1.95 | 0.96 | 0.45 | 1.51 | 1.35 | Mrowietz U, 1999 [23] |
| 271985 | Tyrosinase | 1.87 | 0.98 | 0.48 | 1.39 | 2.46 | Jimenez-Cervantes C, 2001 [24] |
| 450375 | Coagulation factor VIII-associated | 1.81 | 0.47 | 1.01 | 2.26 | 2.57 | |
| 222181 | ATPase, Ca++ transporting, cardiac muscle, slow twitch 2 | 1.76 | 1.05 | 0.46 | 1.48 | 2.47 | |
| 133178 | Yamaguchi sarcoma viral oncogene homolog 1 | 0.45 | 1.43 | 1.34 | 0.47 | 0.48 | Loganzo F, 1993 [14] |
| 452374 | Orosomucoid 1 | 0.44 | 1.16 | 1.64 | 0.59 | 0.48 | |

1. A method for identifying one or more biomarker genes for a first type of cells among a group of m different types of cells, from a multiplicity of genes whose expression levels in cells of said group are measured using one or more nucleic acid arrays, thereby generating a plurality of measurements of expression levels for said m types of cells, which method comprises:
(a) calculating, for each gene, a likelihood ratio in said first type of cells by dividing (i) the product of (m−1) and said measurement for said first type of cells by (ii) the sum of said measurements for said m types of cells excluding the measurement for said first type of cells;
(b) repeating step (a) for (m−1) times to calculate, for said each gene, a likelihood ratio in each of said m types of cells excluding said first type of cells, thereby obtaining (m−1) likelihood ratios for said gene; and
(c) comparing said likelihood ratio of step (a) with said (m−1) likelihood ratios of step (b) for each gene and thereby determining a rank order for said each gene among said multiplicity,
wherein said one or more biomarker genes are identified from said rank order.

2. The method of claim 1, wherein a natural logarithm is taken for each likelihood ratio calculated for each gene in each type of cells in the group and the natural logarithm is designated as the Gibbs likelihood for said each gene, wherein said rank order is determined according to said Gibbs likelihood for said each gene among said multiplicity.

3. The method of claim 2, wherein the comparing further comprises ordering said each gene by said Gibbs likelihoods, or sum of the Gibbs likelihoods for said each gene in the m types of cells, or average of the Gibbs likelihoods for said each gene in the m types of cells, thereby generating a Gibbs gene expression rank, wherein said rank order is determined based on said Gibbs gene expression rank.

4. The method of claim 3, wherein an arithmetic mean of the Gibbs likelihoods for said genes in the multiplicity is taken and a standard deviation of said Gibbs likelihoods in the m types of cells is assessed, wherein the Gibbs likelihoods for said each gene in the first type of cells is represented in the units of said standard deviation plus or minus the corresponding arithmetic mean thereby determining a rank for said each gene in said rank order.

5. The method of claim 4, wherein one or more genes with a Gibbs likelihood greater than u times said standard deviation are designated as up-regulated biomarker genes of said first type of cells.

6. The method of claim 5, wherein u is greater than 1.

7. The method of claim 5, wherein u equals 2.

8. The method of claim 4, wherein one or more genes with a Gibbs likelihood ratio smaller than v times said standard deviation are designated as down-regulated biomarker genes of said first type of cells.

9. The method of claim 8, wherein v is greater than 1.

10. The method of claim 8, wherein v equals 2.

11. The method of claim 1, wherein a median is taken for the likelihood ratios calculated for each gene in the m types of cells, said median being designated as median likelihood, wherein said rank order is determined according to said median likelihood for said each gene among said multiplicity.

12. The method of claim 11, wherein the comparing further comprises generating a median rank distribution by sorting the genes in said multiplicity according to the corresponding median likelihoods, wherein said rank order is determined based on said median gene expression rank.

13. The method of claim 12, wherein an arithmetic mean of the median likelihoods for said genes in the multiplicity is taken and a standard deviation of said median likelihoods in the m types of cells is assessed, wherein the median likelihoods for said each gene in the first type of cells is represented in the units of said standard deviation plus or minus the corresponding arithmetic mean thereby determining a rank for said each gene in said rank order.

14. The method of claim 13, wherein one or more genes with a median likelihood greater than u times said standard deviation are designated as down-regulated biomarker genes of said first type of cells.

15. The method of claim 14, wherein u is greater than 1.

16. The method of claim 14, wherein u equals 2.

17. The method of claim 14, wherein one or more genes with a median likelihood ratio smaller than v times said standard deviation are designated as up-regulated biomarker genes of said first type of cells.

18. The method of claim 17, wherein v is greater than 1.

19. The method of claim 17, wherein v equals 2.

20. The method of claim 1, wherein m is greater than or equals 3.

21. The method of claim 1, wherein the different types of cells are cells or tissues that are normal or abnormal.

22. The method of claim 1, wherein the different types of cells may be exposed to one or more of the treatments selected from the group consisting of treatments with a chemical, a drug, a toxin, a biological agent, an environmental stimulus, and combinations thereof.

23. The method of claim 22, wherein the environmental stimulus comprises electromagnetic radiation, heat, mechanical force, or a combination thereof.

24. The method of claim 1, wherein the different types of cells are skin cells.

25. The method of claim 24, wherein said skin cells comprise keratinocyte cells, melanocyte cells, fibroblast cells or combinations thereof.

26. The method of claim 24, wherein said skin cells comprise melanocyte cells, cutaneous primary melanoma cells, metastatic melanoma cells, or combinations thereof.

27. A gene selected from the group consisting of transducer of ERBB2 member 2, Finkel-Biskis-Reilly murine sarcoma virus, RAB6, homeobox A10, Tax1 binding protein 1, SET binding factor 1, maternally expressed 3, ubiquitination factor E4A, solute carrier family 1 member 3, solute carrier family 2 member 4, heterogeneous nuclear ribonucleoprotein A3, hemogen, apolipoprotein D, cartilage linking protein 1, RNA helicase-related protein, hippocalcin, dystrobrevin alpha, coagulation factor C homolog, putative receptor protein, mitochondrial ornithine transporter, cyclin G2, EST cDNA ID 471826, EST cDNA ID 427657, EST cDNA ID 298104, EST cDNA ID 1571632, EST cDNA ID 591143, and EST cDNA ID 208082 as set forth in Table 10, which gene is an up-regulated biomarker of metastatic melanoma cells.

28. A gene selected from the group consisting of histidyl-tRNA synthetase homolog and an EST cDNA ID 209841 as set forth in Table 9, which gene is an up-regulated biomarker of primary cutaneous melanoma cells.

29. A gene selected from the group consisting of hypothetical protein expressed in osteoblasts, nidogen 2, erythroid alpha-spectrin 1, afx1 transcription factor, and sarcoma-amplified sequence, visinin-like 1, checkpoint suppressor 1, putative nuclear protein, ephrin-B1, biglycan, protein tyrosine phosphatase IVA member 2, prostaglandin E synthase, mitogen-activated protein kinase 10, methylenetetrahydrofolate dehydrogenase, mitochondrial F1 alpha 1 ATP synthase, peroxisomal biogenesis factor 12, pleiomorphic adenoma gene 1, HLA class II region expressed gene K4, coagulation factor VIII-associated, and cardiac muscle slow twitch 2 ATPase, which gene is an up-regulated biomarker of melanocytes.

30. A gene selected from the group consisting of keratin 1, fibroblast growth factor 12, intercellular adhesion molecule 2, hematopoietic protein 1, nuclear domain 10, interleukin-1 receptor-associated kinase, and macrophage associated antigen, which gene is a down-regulated biomarker gene for metastatic melanoma cells.

31. A gene selected from the group consisting of small proline-rich protein 2C, type VIII alpha 1 collagen, type IV alpha 4 collagen, trophinin, chondroitin sulfate proteoglycan 3, activin A receptor type II-like 1, paired box gene 6, homeobox D4, homeobox B5, zinc finger protein 131, special AT-rich sequence binding 1, ubiquitin specific protease 16, pyrolin-5-carboxylate synthetase, neural expressed developmentally down-regulated 5, ribonuclease P (30 kD), protein tyrosine phosphatase (rec F), endothelial lipase, ras homolog gene, valyl-tRNA synthetase 2, arylsulfatase A, aldo-keto reductase 1C1, protein phosphatase 1 regulatory 3C, developmentally regulated GTP-binding 1, 3-hydroxybutyrate dehydrogenase, adipose most abundant transcript, pancreatic polypeptide 2, solute carrier 11A2, solute carrier 22A11, cardiac ankyrin repeat protein, heparin-binding growth factor binding protein, Ewing sarcoma break point region 1, and EST cDNA ID 415281, EST cDNA ID 460258, EST cDNA ID 415235, EST cDNA ID 67330, EST cDNA ID 460247, EST cDNA ID 1522679, EST cDNA ID 378420, EST cDNA ID 341317, EST cDNA ID 461287, EST cDNA ID 415613 as set forth in Table 3, which gene is an up-regulated biomarker for keratinocytes.

32. A gene selected from the group consisting of fibulin 5, interleukin 2 receptor gamma, eukaryotic translation elongation factor 2, mitochondrial ribosomal protein L23, ribosomal protein L7a, SEC23-like protein B, solute carrier family 16A3, metallothionein 1F, metallothionein 1H, interferon induced transmembrane 2, Dickkopf homolog 3, episialin, high mobility group protein I-C, and growth factor receptor-bound protein 14, EST cDNA ID 1049033, and EST cDNA ID 378458 as set forth in Table 2, which gene is an up-regulated biomarker for fibroblasts.

33. A gene selected from the group consisting of galectin 3, syndecan binding protein (syntenin), dystroglycan 1, prostate differentiation factor, glutaminyl cyclotransferase, Na+/K+ transporting ATPase alpha 1, cAMP-dependent protein kinase I alpha 1, protein tyrosine phosphatase IVA 2, fyn oncogene, 6-pyruvoyl-tetrahydropterin synthase, dihydopyrimidinase, pirin, major histocompatibility complex I-C, 4F2 antigen heavy chain (solute carrier 3), abl-interactor 2b, coxsackie virus and adenovirus receptor, prostatic binding protein, proteolipid protein 1, v-abl oncogene 1, ets2 repressor factor, proline-rich Gla 1, axin 1 up-regulated, voltage-gated K+ channel beta subunit, vaccinia-related kinase 3, EST cDNA ID 712604, EST cDNA ID 267859, EST cDNA ID 320588, EST cDNA ID 305843, as set forth in Table 3, which gene is an up-regulated biomarker of melanocytes.

34. A gene selected from the group consisting of MIC2 (antigen to antibodies 12E7, F21, and O13), microtubule-associated protein 1B, monocytic leukemia zinc finger protein, Clathrin heavy chain 1, non-metastatic cells 4, TC10-like Rho GTPase, Myclin gene expression factor 2, and CAAX box 1, EST cDNA ID 53371, and EST cDNA ID 1467936 as set forth in Table 4, which gene is a down-regulated biomarker for keratinocytes.

35. A gene selected from the group consisting of long chain 2 of Fatty-acid coenzyme A ligase, calcium modulating ligand, and nuclear receptor coactivator 3 (amplified breast cancer—AIBI), which gene is a down-regulated biomarker for fibroblasts.

36. A gene selected from the group consisting of ribosomal protein L30 and orosomucoid 1, which gene is a down-regulated biomarker for melanocytes.

37. A sequence selected from the group consisting of the genes and sequences identified in Tables 1-11, and combinations thereof, which is a diagnostic biomarker for a mammal.

38. The sequence of claim 37, wherein the mammal is a human.

39. The sequence selected from the group consisting of the genes and sequences identified on Tables 1-11, and combinations thereof, which is a molecular target for therapeutics of a mammalian disorder or for the discovery of therapeutics of a mammalian disorder.

40. The sequence of claim 39, wherein the mammalian disorder is a human disorder.
